SEQUENCE LISTING (23 sequences)

SEQ ID NO: 1 - DNA, 1071 bp, Oryza sativa
  CDS (1)..(1071)
atggcgtcgctcaccgccgccctggccacgccggcggccgctgccctcctcctcctcgtcctcctcgccgcccccgcctccgccgccaacttcacctgcgcggtggcttcaggcaccacctgcaagtccgccatcctctacacctcccccaacgccaccacctacggcaacctcgtcgcccgcttcaacaccaccaccctccccgacctcctcggcgccaacggcctccccgacggcacgctttcctccgcccccgtcgccgccaattccaccgtcaaaatccccttccgctgccgctgcaacggcgacgtcggccagtcggaccgcctccccatctacgtcgtgcagccgcaggacgggctcgacgccatcgcgcgcaacgtgttcaacgccttcgtcacctaccaggagatcgccgccgcgaacaacatccccgaccccaacaagataaatgtcagccagacgctgtggattccgctgccctgcagctgcgacaaggaggaaggctctaacgtgatgcacctcgcctacagcgtcggcaaaggggagaacacgtcggcgatcgctgccaagtacggggtgacggagtccacgcttctcaccagaaataagatcgacgaccccacgaaattgcagatgggacagattctagatgtcccgctccctgtgtgccgttcatcaatcagcgatacctcagctgatcacaatctgatgctcctcccggatggcacctatggattcaccgcaggaaactgcatccgctgcagctgcagttcaactacctaccagctaaactgcactgcagtacagaacaagggatgcccgtcagtgccactgtgcaatggaacgctgaagcttggtgagacgaacggcaccggttgcggatcaacaacgtgcgcctacagtggttactccaacagttcatcgctcatcatacaaaccagccttgcaactaatcagacaacagcctgccagagaggaggatctgggaggtcgcagttcgctaggtccatgtggagcatgtctgttatctccttccacatggtgttgatcattatctgtttcctttga

SEQ ID NO: 2 - protein, 356 aa, Oryza sativa
MASLTAALATPAAAALLLLVLLAAPASAANFTCAVASGTTCKSAILYTSPNATTYGNLVARFNTTTLPDLLGANGLPDGTLSSAPVAANSTVKIPFRCRCNGDVGQSDRLPIYVVQPQDGLDAIARNVFNAFVTYQEIAAANNIPDPNKINVSQTLWIPLPCSCDKEEGSNVMHLAYSVGKGENTSAIAAKYGVTESTLLTRNKIDDPTKLQMGQILDVPLPVCRSSISDTSADHNLMLLPDGTYGFTAGNCIRCSCSSTTYQLNCTAVQNKGCPSVPLCNGTLKLGETNGTGCGSTTCAYSGYSNSSSLIIQTSLATNQTTACQRGGSGRSQFARSMWSMSVISFHMVLIIICFL

SEQ ID NO: 3 - DNA, 987 bp, Oryza sativa
  CDS (1)..(987)
gccaacttcacctgcgcggtggcttcaggcaccacctgcaagtccgccatcctctacacctcccccaacgccaccacctacggcaacctcgtcgcccgcttcaacaccaccaccctccccgacctcctcggcgccaacggcctccccgacggcacgctttcctccgcccccgtcgccgccaattccaccgtcaaaatccccttccgctgccgctgcaacggcgacgtcggccagtcggaccgcctccccatctacgtcgtgcagccgcaggacgggctcgacgccatcgcgcgcaacgtgttcaacgccttcgtcacctaccaggagatcgccgccgcgaacaacatccccgaccccaacaagataaatgtcagccagacgctgtggattccgctgccctgcagctgcgacaaggaggaaggctctaacgtgatgcacctcgcctacagcgtcggcaaaggggagaacacgtcggcgatcgctgccaagtacggggtgacggagtccacgcttctcaccagaaataagatcgacgaccccacgaaattgcagatgggacagattctagatgtcccgctccctgtgtgccgttcatcaatcagcgatacctcagctgatcacaatctgatgctcctcccggatggcacctatggattcaccgcaggaaactgcatccgctgcagctgcagttcaactacctaccagctaaactgcactgcagtacagaacaagggatgcccgtcagtgccactgtgcaatggaacgctgaagcttggtgagacgaacggcaccggttgcggatcaacaacgtgcgcctacagtggttactccaacagttcatcgctcatcatacaaaccagccttgcaactaatcagacaacagcctgccagagaggaggatctgggaggtcgcagttcgctaggtccatgtggagcatgtctgttatctccttccacatggtgttgatcattatctgtttcctttga

SEQ ID NO: 4 - protein, 328 aa, Oryza sativa
ANFTCAVASGTTCKSAILYTSPNATTYGNLVARFNTTTLPDLLGANGLPDGTLSSAPVAANSTVKIPFRCRCNGDVGQSDRLPIYVVQPQDGLDAIARNVFNAFVTYQEIAAANNIPDPNKINVSQTLWIPLPCSCDKEEGSNVMHLAYSVGKGENTSAIAAKYGVTESTLLTRNKIDDPTKLQMGQILDVPLPVCRSSISDTSADHNLMLLPDGTYGFTAGNCIRCSCSSTTYQLNCTAVQNKGCPSVPLCNGTLKLGETNGTGCGSTTCAYSGYSNSSSLIIQTSLATNQTTACQRGGSGRSQFARSMWSMSVISFHMVLIIICFL

SEQ ID NO: 5 - DNA, 1612 bp, Oryza sativa
ttccatcggtaattttgaaatttcagtgattttttgttctccaagtctgactcaaaccaatcaattccacacaagttcaccattttctactttgatgcaatcgaaccaaaggatagggaaaggcccacgctgcaaccatttaagtcgccacaagcactagcatcgaggcatactcctcctgctccagtgtcaacacgacagtcaaacaaacaccaagcctctcattcccacctctcgaggagagctttccccaccatggcgtcgctcaccgccgccctggccacgccggcggccgctgccctcctcctcctcgtcctcctcgccgcccccgcctccgccgccaacttcacctgcgcggtggcttcaggcaccacctgcaagtccgccatcctctacacctcccccaacgccaccacctacggcaacctcgtcgcccgcttcaacaccaccaccctccccgacctcctcggcgccaacggcctccccgacggcacgctttcctccgcccccgtcgccgccaattccaccgtcaaaatccccttccgctgccgctgcaacggcgacgtcggccagtcggaccgcctccccatctacgtcgtgcagccgcaggacgggctcgacgccatcgcgcgcaacgtgttcaacgccttcgtcacctaccaggagatcgccgccgcgaacaacatccccgaccccaacaagataaatgtcagccagacgctgtggattccgctgccctgcagctgcgacaaggaggaaggctctaacgtgatgcacctcgcctacagcgtcggcaaaggggagaacacgtcggcgatcgctgccaagtacggggtgacggagtccacgcttctcaccagaaataagatcgacgaccccacgaaattgcagatgggacagattctagatgtcccgctccctgtgtgccgttcatcaatcagcgatacctcagctgatcacaatctgatgctcctcccggatggcacctatggattcaccgcaggaaactgcatccgctgcagctgcagttcaactacctaccagctaaactgcactgcagtacagaacaagggatgcccgtcagtgccactgtgcaatggaacgctgaagcttggtgagacgaacggcaccggttgcggatcaacaacgtgcgcctacagtggttactccaacagttcatcgctcatcatacaaaccagccttgcaactaatcagacaacagcctgccagagaggaggatctgggaggtcgcagttcgctaggtccatgtggagcatgtctgttatctccttccacatggtgttgatcattatctgtttcctttgatgttggagactactgcaactctagatggtacatttcaaagagttctctacgatctatgattgttgtatacgatatatgattgttgtcgtaacttagattttgatgactggtttatccagctttgaaatttgagttttgactctgttctttagaggatgagtggcacttgtacggctgcttgaataaaacgtcgatgtattgtattcgatctgcatctgaaaaggaatattcattagataggattattcgaaataaaaagatcccacatgtttttgttaaaaaaaaa

SEQ ID NO: 6 - protein, 435 aa, Oryza sativa
NFSDFLFSKSDSNQSIPHKFTIFYFDAIEPKDRERPTLQPFKSPQALASRHTPPAPVSTRQSNKHQASHSHLSRRAFPTMASLTAALATPAAAALLLLVLLAAPASAANFTCAVASGTTCKSAILYTSPNATTYGNLVARFNTTTLPDLLGANGLPDGTLSSAPVAANSTVKIPFRCRCNGDVGQSDRLPIYVVQPQDGLDAIARNVFNAFVTYQEIAAANNIPDPNKINVSQTLWIPLPCSCDKEEGSNVMHLAYSVGKGENTSAIAAKYGVTESTLLTRNKIDDPTKLQMGQILDVPLPVCRSSISDTSADHNLMLLPDGTYGFTAGNCIRCSCSSTTYQLNCTAVQNKGCPSVPLCNGTLKLGETNGTGCGSTTCAYSGYSNSSSLIIQTSLATNQTTACQRGGSGRSQFARSMWSMSVISFHMVLIIICFL

SEQ ID NO: 7 - DNA, 918 bp, Oryza sativa
  CDS (1)..(918)
gccaacttcacctgcgcggtggcttcaggcaccacctgcaagtccgccatcctctacacctcccccaacgccaccacctacggcaacctcgtcgcccgcttcaacaccaccaccctccccgacctcctcggcgccaacggcctccccgacggcacgctttcctccgcccccgtcgccgccaattccaccgtcaaaatccccttccgctgccgctgcaacggcgacgtcggccagtcggaccgcctccccatctacgtcgtgcagccgcaggacgggctcgacgccatcgcgcgcaacgtgttcaacgccttcgtcacctaccaggagatcgccgccgcgaacaacatccccgaccccaacaagataaatgtcagccagacgctgtggattccgctgccctgcagctgcgacaaggaggaaggctctaacgtgatgcacctcgcctacagcgtcggcaaaggggagaacacgtcggcgatcgctgccaagtacggggtgacggagtccacgcttctcaccagaaataagatcgacgaccccacgaaattgcagatgggacagattctagatgtcccgctccctgtgtgccgttcatcaatcagcgatacctcagctgatcacaatctgatgctcctcccggatggcacctatggattcaccgcaggaaactgcatccgctgcagctgcagttcaactacctaccagctaaactgcactgcagtacagaacaagggatgcccgtcagtgccactgtgcaatggaacgctgaagcttggtgagacgaacggcaccggttgcggatcaacaacgtgcgcctacagtggttactccaacagttcatcgctcatcatacaaaccagccttgcaactaatcagacaacagcctgccagagaggaggatctgggaggtcgcagttc

SEQ ID NO: 8 - protein, 306 aa, Oryza sativa
ANFTCAVASGTTCKSAILYTSPNATTYGNLVARFNTTTLPDLLGANGLPDGTLSSAPVAANSTVKIPFRCRCNGDVGQSDRLPIYVVQPQDGLDAIARNVFNAFVTYQEIAAANNIPDPNKINVSQTLWIPLPCSCDKEEGSNVMHLAYSVGKGENTSAIAAKYGVTESTLLTRNKIDDPTKLQMGQILDVPLPVCRSSISDTSADHNLMLLPDGTYGFTAGNCIRCSCSSTTYQLNCTAVQNKGCPSVPLCNGTLKLGETNGTGCGSTTCAYSGYSNSSSLIIQTSLATNQTTACQRGGSGRSQF

SEQ ID NO: 9 - protein, 32 aa, Oryza sativa
  MISC_FEATURE (2)..(2): “Xaa” indicates any amino acid.
  MISC_FEATURE (5)..(5): “Xaa” indicates any amino acid.
  MISC_FEATURE (13)..(13): “Xaa” indicates any amino acid.
  MISC_FEATURE (30)..(30): “Xaa” indicates any amino acid.
AXFTXAVASGTTXKSAILYTSPVATTYGNXVA

SEQ ID NO: 10 - protein, 7 aa, Oryza sativa
TYLIASK

SEQ ID NO: 11 - DNA, 19 bp, Artificial
  An artificially synthesized primer sequence
tgtagaggatggcggactt

SEQ ID NO: 12 - DNA, 19 bp, Artificial
  An artificially synthesized primer sequence
ggaaacagctatgaccatg

SEQ ID NO: 13 - DNA, 385 bp, Oryza sativa
acagaacaagggatgcccgtcagtgccactgtgcaatggaacgctgaagcttggtgagacgaacggcaccggttgcggatcaacaacgtgcgcctacagtggttactccaacagttcatcgctcatcatacaaaccagccttgcaactaatcagacaacagcctgccagagaggaggatctgggaggtcgcagttcgctaggtccatgtggagcatgtctgttatctccttccacatggtgttgatcattatctgtttcctttgatgttggagactactgcaactctagatggtacatttcaaagagttctctacgatctatgattgttgtatacgatatatgattgttgtcgtaacttagattttgatgactggtttatccagc

SEQ ID NO: 14 - DNA, 24 bp, Artificial
  an artificially synthesized primer sequence
caccacagaacaagggatgcccgt

SEQ ID NO: 15 - DNA, 24 bp, Artificial
  an artificially synthesized primer sequence
gctggataaaccagtcatcaaaat

SEQ ID NO: 16 - DNA, 21 bp, Artificial
  an artificially synthesized primer sequence
caccctacagtggttactcca

SEQ ID NO: 17 - DNA, 20 bp, Artificial
  an artificially synthesized primer sequence
tcctatctaatgaatattcc

SEQ ID NO: 18 - DNA, 20 bp, Artificial
  an artificially synthesized primer sequence
tacccgcttcgcgtcggcat

SEQ ID NO: 19 - DNA, 20 bp, Artificial
  an artificially synthesized primer sequence
tgcttccgccagtggcgcga

SEQ ID NO: 20 - DNA, 22 bp, Artificial
  an artificially synthesized primer sequence
ccagtaagtcctcagccatgga

SEQ ID NO: 21 - DNA, 22 bp, Artificial
  an artificially synthesized primer sequence
ggacacaatgattagggatcac

SEQ ID NO: 22 - DNA, 24 bp, Artificial
  an artificially synthesized primer sequence
atggaacgctgaagcttggtgaga

SEQ ID NO: 23 - DNA, 24 bp, Artificial
  an artificially synthesized primer sequence
ctcatcctctaaagaacagagtca

(19) United States
(12) Patent Application Publication
(10) Pub. No.: US 2007/0275464 A1
(43) Pub. Date: Nov. 29, 2007

(54) Chitin Oligosaccharide Elicitor-Binding Proteins

(75) Inventors: Hanae Kaku, Ibaraki (JP); Naoto Shibuya, Kanagawa (JP); Eiichi Minami, Ibaraki (JP); Naoko Minami, Ibaraki (JP); Yoko Nishizawa, Ibaraki (JP); Koji Takio, Nara (JP); Naoshi Dohmae, Saitama (JP)

Correspondence Address: MEDLEN & CARROLL, LLP, 101 HOWARD STREET, SUITE 350, SAN FRANCISCO, CA, 94105, US

(73) Assignee: NATIONAL INSTITUTE OF AGROBIOLOGICAL SCIENCES, Tsukuba-shi, Ibaraki (JP)

(21) Appl. No.: 10/591,576

(22) PCT Filed: Mar. 2, 2005
(86) PCT No.: PCT/JP05/03451

§ 371 Date: May 21, 2007

(30) Foreign Application Priority Data
Mar. 3, 2004 (JP) 2004-059551

(51) Int. Cl. A01H 5/00; A01N 43/04; C07H 21/04; C07K 14/00; C12N 15/00; C12N 15/87; C12N 5/00

(52) U.S. Cl. 435/410; 435/320.1; 435/430; 514/44; 530/350; 536/23.6; 800/279; 800/320.2

(57) ABSTRACT

The present inventors isolated and purified elicitor-binding proteins in good yield by combining the development of a column that uses APEA derivatives, pre-columns to remove non-specifically adsorbing substances, and effective elution methods. Using the N-terminal and internal chain amino acid sequences of the obtained proteins, the present inventors successfully isolated cDNAs encoding the proteins of the present invention from a rice cDNA library. Moreover, when anti-Con A-CEBiP antibodies were purified and their effect on elicitor-responsive reactive oxygen production was examined, production of reactive oxygen was inhibited by a pretreatment with the antibodies, suggesting that the present proteins are receptor proteins involved in chitin oligosaccharide elicitor responses. Since these elicitors induce resistance to blast in rice, the proteins of the present invention can be applied to the development of novel disease control technologies.

### TECHNICAL FIELD

The present invention relates to proteins that bind to chitin oligosaccharide elicitors.

### BACKGROUND ART

Identifying receptor molecules is one of the most important objectives in elucidating molecular mechanisms of biological defense signal-transduction processes induced by elicitors in plants; however, there are very few examples of plant cell membrane-bound receptors identified together with the relationship to their corresponding ligands. Soybean glucan-type elicitor-binding protein is the most thoroughly examined of the elicitor receptors so far, and a cDNA that conceivably encodes this elicitor-binding protein in the plasma membrane has been cloned. However, since in this case also the protein structure deduced from the cDNA does not have a typical receptor-like structure, its functional role in signal-transduction processes is still mostly unknown. Recently, a receptor kinase gene (FLS2) assumed to be a flagellin elicitor receptor has been isolated by molecular genetic techniques, but whether this gene product itself binds with the elicitor has not been determined. In terms of other examples, cases where binding proteins have been identified are limited, and there are no reports regarding the identity of chitin-type elicitors other than those from studies conducted by the present inventors.

So far, the present inventors revealed that in cultured rice cells, specifically sized fragments of chitin, a filamentous fungi's cell wall-constituting polysaccharide, can induce phytoalexin synthesis, membrane depolarization, ion influx and efflux, protein phosphorylation, reactive oxygen production, synthesis of jasmonic acid, and gene expression of chitinase, PAL (phenylalanine ammonia liase), and such at low concentrations such as in the order of nanomolars (see Non-Patent Documents 1 to 6). The present inventors have also demonstrated that the level of these cellular responses is decreased when using chitosan oligosaccharides, which are deacetylated forms of this elicitor, and chitin oligosaccharides whose degree of polymerization is five or less. These facts indicate that a receptor that strictly recognizes the size and structure of chitin oligosaccharides is present in cultured rice cells. The present inventors have confirmed the presence of a high-affinity elicitor-binding protein in the plasma membrane fraction of rice from experiments using ²⁵I-labeled chitin oligosaccharides, and they have also used biochemical analyses to confirm its binding specificity to cultured cells, which corresponded well with the elicitor activity (see Non-Patent Documents 7 to 9).

Prior art literature relating to the invention of the present application is shown below:
[Non-Patent Document 1] Yamada, A., Shibuya, N., Kodama, O. and Akatsuka, T.: Induction of phytoalexin formation in suspension-cultured rice cells by N-acetylchitooligosaccharides, Biosci. Biotechnol. Biochem., 57, 405-409 (1993).
[Non-Patent Document 2] Minami, E., Kuchitsu, K., He, D.-Y., Kouchi, H., Midoh, N., Ohtsuki, Y. and Shibuya, N.: Two novel genes rapidly and transiently activated in suspension-cultured rice cells by treatment with N-acetylchitoheptaose, a biotic elicitor for phytoalexin production. Plant Cell Physiol., 37, 563 (1996).
[Non-Patent Document 3] Kikuyama, M., Kuchitsu, K. and Shibuya, N.: Membrane depolarization induced by N-acetylchitooligosaccharide elicitor in suspension-cultured rice cells. Plant Cell Physiol. 38, 902-909 (1997).
[Non-Patent Document 4] He, D.-Y., Yazaki, Y, Nishizawa, Y., Takai, R., Yamada, K., Sakano, K., Shibuya, N. and Minami, E. Gene activation by cytoplasmic acidification in suspension-cultured rice cells in response to the potent elicitor, N-acetylchitoheptaose. Mol. Plant-Microbe. Interact., 11, 1167 (1998).
[Non-Patent Document 5] R. Takai, K. Hasegawa, H. Kaku, N. Shibuya and E. Minami:Isolation and analysis of expression mechanisms of a rice gene, EL5, which shows structural similarity to ATL family from Arabidopsis, in response to N-acetylchitooligosaccharide elicitor. Plant Science, 160, 577-583 (2001).
[Non-Patent Document 6] Yamaguchi, T., Minami, E. and Shibuya, N.: Activation of phospholipases by N-acetylchitooligosaccharide elicitor in suspension-cultured rice cells mediates reactive oxygen generation, Physiol. Plant., 118, 361-370 (2003).
[Non-Patent Document 7] Shibuya, N., Kaku, H., Kuchitsu, K., and Maliarik, M. J.: Identification of a novel high-affinity binding site for N-acetylchitooligosaccharide elicitor in the membrane fraction from suspension-cultured rice cells. FEBS Lett., 329, 75-78 (1993).
[Non-Patent Document 8] Shibuya, N., Ebisu, N., Kamada, Y, Kaku, H., Cohn, J. and Ito, Y.: Localization and binding characteristics of a high-affinity binding site for N-acetylchitooligosaccharide in the plasma membrane from suspension-cultured rice cells suggest a role as a receptor for the elicitor signal at the cell surface. Plant Cell Physiol., 37, 894-898 (1996).
[Non-Patent Document 9] Ito, Y, Kaku, H. and Shibuya N.: Identification of a high-affinity binding protein for N-acetylchitooligosaccharide elicitor in the plasma membrane of suspension-cultured rice cells by affinity labeling. The Plant Journal, 12(2), 347-356 (1997).

### DISCLOSURE OF THE INVENTION

### Problems to be Solved by the Invention

An objective of the present invention is to provide proteins that bind to chitin oligosaccharide elicitors and methods for using such proteins.

### Means to Solve the Problems

The present inventors conducted dedicated research to solve the above-mentioned problems. Methods for purifying chitin oligosaccharide elicitor-binding proteins by using columns to which chitin oligosaccharides were immobilized, while maintaining the proteins' activity to bind to chitin oligosaccharide elicitors, required a variety of investigations on: the setting of conditions for solubilizing membrane proteins; the design of pre-columns to avoid contamination with non-specifically adsorbing proteins and the like; and the design of affinity supports and setting of elution conditions that increase the adsorption capacities and recovery rates. Specifically, elicitor-binding proteins were isolated and purified with good yields by combining: a solubilization of plasma membrane proteins using Triton X-100; the development of a column using APEA derivatives ((GlcNAc)₈-APEA (aminophenylethylamino) derivatives); pre-columns for removing non-specifically adsorbing substances; and effective elution methods. Next, the N-terminal and internal chain amino acid sequences were clarified, and cDNAs encoding the proteins of the present invention were successfully isolated from rice cDNA libraries based on this amino acid sequence information.

The proteins of the present invention are glycoproteins and bind to Con A lectin. Therefore, antibodies against a rice plasma membrane fraction that binds to a Con A column were produced, various chromatographies were devised, and antibodies against the present target protein (anti-Con A-CEBiP antibodies) were purified. When the effect of anti-ConA-CEBiP antibodies on the elicitor-responsive reactive oxygen production was examined, the production of reactive oxygen was inhibited by a pretreatment with anti-Con A-CEBiP antibodies, suggesting that the proteins of the present invention are receptor proteins involved in chitin oligosaccharide elicitor responses. Transformed rice cells whose CEBiP gene expression had been specifically knocked down by RNAi methods were also subjected to sugar elicitor treatment. As a result, the production of reactive oxygen was suppressed, which also suggested that the proteins of the present invention are receptor proteins involved in chitin oligosaccharide elicitor responses. Furthermore, microarray analyses showed that 70% or more of the genes that respond to chitin oligosaccharide elicitors in cultured rice cells lose their responsiveness when CEBiP expression level is decreased; therefore, CEBiPs are confirmed to be receptor proteins for chitin oligosaccharide elicitors and are proteins with an important role in elicitor signal transduction.

Therefore, the present invention provides the following (1) to (16):

(1) a DNA encoding a plant protein that has a binding activity to a chitin oligosaccharide elicitor, wherein the DNA is any one of (a) to (d):

(a) a DNA comprising the nucleotide sequence of SEQ ID NO: 1 or 3;

(b) a DNA that hybridizes with a DNA comprising the nucleotide sequence of SEQ ID NO: 1 or 3;

(c) a DNA encoding a protein comprising the amino acid sequence of SEQ ID NO: 2 or 4; and

(d) a DNA encoding a protein comprising an amino acid sequence with a substitution, deletion, addition, and/or insertion of one or more amino acids in the amino acid sequence of SEQ ID NO: 2 or 4;
(2) the DNA of (1), wherein the plant is rice;
(3) a protein encoded by the DNA of (1) or (2);
(4) a vector comprising the DNA of (1) or (2);
(5) a transformed plant cell that carries the DNA of (1) or (2), or the vector of (4);
(6) a plant transformant comprising the transformed plant cell of (5);
(7) the plant transformant of (6), which is derived from rice;
(8) a plant transformant that is a progeny or a clone of the plant transformant of (6) or (7);
(9) a breeding material of the plant transformant of any one of (6) to (8);
(10) a method for producing the plant transformant of any one of (6) to (8), wherein the method comprises the steps of introducing the DNA of (1) or (2), or the vector of (4) into a plant cell, and regenerating a plant from the plant cell;
(11) a pharmaceutical agent used to control a plant disease, wherein the agent comprises the DNA of (1) or (2), or the vector of (4);
(12) the pharmaceutical agent of (11), wherein the plant is rice;
(13) the pharmaceutical agent of (12), wherein the disease is blast;
(14) a method for controlling a plant disease, wherein the method comprises the step of expressing the protein of (3) in a cell of a plant;
(15) the method of (14), wherein the plant is rice; and
(16) the method of (15), wherein the disease is blast.

### BRIEF DESCRIPTION OF THE DRAWINGS

FIG. 1 is a diagram and a photograph showing the purification of chitin elicitor-binding proteins from cultured rice cells.

FIG. 2 shows a method for purifying anti-Con A-CEBiP antibodies.

FIG. 3 shows photographs of a two-dimensional SDS-PAGE of a rice plasma membrane fraction that binds to affinity-labeled Con A.

FIG. 4 is a graph showing the effect of anti-CEBiP antibodies or antisera on reactive oxygen production.

FIG. 5 is a diagram showing a 32-residue N-terminal amino acid sequence obtained from the peptide sequencer.

FIG. 6 is a schematic diagram of a procedure for cloning oligochitin-binding proteins.

FIG. 7 is a diagram showing a cDNA of a rice-derived chitin elicitor-binding protein. The nucleotide sequence and amino acid sequence in this figure are shown in SEQ ID NOs: 5 and 6, respectively.

FIG. 8 is a photograph showing the results of genomic Southern blot analysis.

FIG. 9 shows the results of matching the sequence of the chitin elicitor-binding protein to a genomic DNA sequence.

FIG. 10 is a photograph showing the effect of (GlcNAc)₇ on CEBiP expression in solubilized cultured cells.

FIG. 11 shows the results of measuring the molecular weight of CEBiP using MALDI TOF-MS.

FIG. 12 is a photograph showing the deglycosylation of CEBiP by TFMS.

FIG. 13 is a diagram showing the nucleotide sequence of the CEBiP gene fragment in the CEBiP-RNAi vector.

FIG. 14 is photographs of protein- and RNA-level expression analyses of the CEBiP gene in non-transformed and transformed rice cells.

FIG. 15 is a graph showing the production of reactive oxygen due to an elicitor in non-transformant and CEBiP-RNAi rice cells.

FIG. 16 is a graph showing the production of reactive oxygen due to elicitor treatment in non-transformant and CEBiP-RNAi rice cells.

FIG. 17 (A) shows graphs indicating the number of genes whose expression levels increased or decreased due to elicitor treatment in non-transformant and CEBiP-RNAi #6 rice cells. (B) shows graphs indicating a function-based classification of genes that were significantly suppressed as a result of knocking down the CEBiP gene.

### BEST MODE FOR CARRYING OUT THE INVENTION

The present inventors isolated and purified a chitin oligosaccharide elicitor-binding protein (CEBiP) found in plants with a high yield, and identified its genetic sequence.

The present invention provides DNAs encoding plant proteins that have a binding activity to chitin oligosaccharide elicitors, wherein the DNAs are any one of (a) to (d), as well as proteins encoded by these DNAs:
(a) DNAs comprising the nucleotide sequence of SEQ ID NO: 1 or 3;
(b) DNAs that hybridize with DNAs comprising the nucleotide sequence of SEQ ID NO: 1 or 3;
(c) DNAs encoding proteins comprising the amino acid sequence of SEQ ID NO: 2 or 4; and
(d) DNAs encoding proteins comprising an amino acid sequence with a substitution, deletion, addition, and/or insertion of one or more amino acids in the amino acid sequence of SEQ ID NO: 2 or 4.

Examples of the above-mentioned plant of the present invention comprise, without limitation, valuable agricultural products such as cereals, vegetables and fruit trees, and ornamental plants such as foliage plants, etc. Specifically, examples of these plants can comprise rice, corn, wheat, barley, rapeseed, soybeans, cotton, carrots, tomatoes, potatoes, chrysanthemums, roses, carnations, cyclamens, Arabidopsis thaliana, and such. The above-mentioned plant of the present invention preferably comprises rice.

The nucleotide sequence of the cDNA of the elicitor-binding protein of the present invention is presented in SEQ ID NO: 1, and SEQ ID NO: 2 shows the amino acid sequence of the protein encoded by this cDNA. Moreover, the nucleotide sequence of the DNA in which the portion encoding a signal peptide has been removed from the cDNA is shown in SEQ ID NO: 3, and the amino acid sequence of the protein encoded by this DNA is presented in SEQ ID NO: 4. The proteins of the present invention comprise four types of internal sequence (the sequence from position 139 to 152, the sequence from position 154 to 161, the sequence from position 164 to 176, the sequence from position 177 to 182 of SEQ ID NO: 4 in the Sequence Listing).

The DNAs of the present invention can be isolated using methods typically known to one skilled in the art. These methods comprise, for example, methods using hybridization techniques (Southern, E M., J. Mol. Biol., 1975, 98, 503) or Polymerase Chain Reaction (PCR) techniques (Saiki, R K. et al., Science, 1985, 230, 1350; Saiki, R K. et al., Science, 1988, 239, 487). Thus, DNAs with high homology to a DNA that comprises the nucleotide sequence of SEQ ID NO: 1 or 3 can generally be isolated from another plant by one skilled in the art. This can be achieved by using, as a probe, a DNA comprising the nucleotide sequence of SEQ ID NO: 1 or 3, or one part of this DNA, and using as primers, oligonucleotides that specifically hybridize to a DNA comprising the nucleotide sequence of SEQ ID NO: 1 or 3. Thus, DNAs of the present invention also comprise DNAs that hybridize to a DNA comprising the nucleotide sequence of SEQ ID NO: 1 or 3, which can be isolated using hybridization or PCR technology in this way.

To isolate this kind of DNA, a hybridization reaction is preferably carried out under conditions of low stringency. Low stringency hybridization conditions (mild hybridization conditions) in the present invention refer to 5×SSPE, 1% SDS (w/v), 0.1% BSA, 0.1% polyvinyl pyrrolidone, 0.1% Ficoll 400, 100 μg/ml denatured salmon sperm DNA, or to hybridization conditions with similar stringency to these. Furthermore, high stringency hybridization conditions, (for example, 25% formaldehyde (v/v), 5×SSPE, 1% SDS (w/v), 0.1% BSA, 0.1% polyvinyl pyrrolidone, 0.1% Ficoll 400, 100 μg/ml denatured salmon sperm DNA) are expected to facilitate the isolation of DNA with greater homology. The DNA isolated in this way is thought to have high homology at the amino acid level with the amino acid sequence of SEQ ID NO: 2 or 4. “High homology” with the full length amino acid sequence means a sequence identity of at least 35%, 40%, 50%, 60%, 70%, 80%, 90%, 95%, or 98% or more.

Identity in amino acid or nucleotide sequences can be determined using Karlin and Altschul's BLAST algorithm (Proc. Natl. Acad. Sci. USA, 1990, 87, 2264-2268; Karlin, S. & Altschul, S F., Proc. Natl. Acad. Sci. USA, 1993, 90, 5873). Programs called BLASTN and BLASTX have been developed using the BLAST algorithm as a base (Altschul, SF. et al., J. Mol. Biol., 1990, 215, 403). When using BLASTN to analyze nucleotide sequences, the parameters can be set at, for example, score=100 and word length=12. In addition, when using BLASTX to analyze amino acid sequences, the parameters can be set at, for example, score=50 and word length=3. When using BLAST and the Gapped BLAST program, the default parameters for each program are used. Specific techniques for these analysis methods are in the well known (http://www.ncbi.nln.nih.gov/).

The present invention also provides proteins structurally similar to the elicitor-binding proteins of the present invention, and DNAs encoding these proteins. Examples of DNAs that encode such proteins comprise DNAs encoding proteins that comprise an amino acid sequence with a substitution, deletion, addition, and/or insertion of one or more amino acids in the amino acid sequence of said proteins. Examples are DNAs encoding glycoproteins which are homologs of an elicitor-binding protein of the present invention, comprising a site that can bind to sugar chains (NXT(S)), and comprising a LysM domain.

In preparing the above DNAs, one skilled in the art could use well known methods, such as the above-mentioned hybridization technique (Southern, E M., J. Mol. Biol., 1975, 98, 503) or PCR technique (Saiki, R K. et al., Science, 1985, 230, 1350; Saiki, R K. et al., Science, 1988, 239, 487). In addition to these methods, techniques such as, for example, introducing mutations using site-directed mutagenesis (Kramer, W. & Fritz, H J., Methods Enzymol., 1987, 154, 350) are also comprised. The number of modified amino acids is not particularly limited, as long as the modified protein is structurally similar to an elicitor-binding protein of the present invention, and the number is generally not more than 50 amino acids, preferably not more than 30 amino acids, and more preferably not more than 10 amino acids (for example, not more than five amino acids, or not more than three amino acids). The amino acid modifications are preferably conservative substitutions. The hydropathic index (Kyte and Doolitte, (1982) J. Mol. Biol 1982 May 5; 157(1):105-32) and hydrophilicity value (U.S. Pat. No. 4,554,101) for each of the amino acids before and after modification are preferably ±2 or less, more preferably ±1 or less, and most preferably ±0.5 or less. In the natural world, mutations in the nucleotide sequence can cause mutations in the amino acid sequence of the coded protein. In some cases, a nucleotide sequence mutation will not lead to a mutation in the amino acid sequence of the protein (a degenerate mutation). Such degenerately mutated DNAs are also comprised in the present invention.

The DNAs of the present invention comprise genomic DNAs, cDNAs and chemically synthesized DNAs. Genomic DNAs and cDNAs can be prepared by practices well used by those skilled in the art. For example, genomic DNAs can be prepared as follows: genomic DNAs are extracted from plants comprising genes coding for the above-mentioned plant elicitor-binding proteins, a genomic library is constructed (a plasmid, phage, cosmid, BAC, PAC or the like can be used as the vector) and developed, and colony or plaque hybridization can be carried out using probes prepared by using a DNA coding for an above-mentioned protein as a base. Alternatively, genomic DNAs can also be prepared by constructing primers specific to a DNA coding for an above-mentioned plant elicitor-binding protein, and then using these primers to carry out PCR. cDNAs can be prepared, for example, as follows: cDNAs are synthesized based on mRNAs extracted from plants which comprise a gene coding for one of the above-mentioned proteins, these cDNAs are inserted into λZAP vector or such, a cDNA library is prepared and developed, and a colony or plaque hybridization, or PCR is carried out in the same way as above.

The naturally derived DNAs of the present invention are induced by chitin oligosaccharide elicitors. Therefore, the induction of the DNAs of the present invention can be used as an index to judge whether a biological defense mechanism is functioning in test plants or test plant cells.

The DNAs of the present invention can be used to produce plant transformants whose phenotypes are modified by regulated expression of these DNAs. Moreover, the DNAs of the present invention can be used, for example, to prepare recombinant proteins. Elicitors are important substances in studying the molecular mechanisms of biological defense signal-transduction processes in plants.

Recombinant proteins are usually prepared by inserting a DNA of the present invention into an appropriate expression vector, introducing the vector into an appropriate cell, culturing the transformed cells, and purifying the expressed proteins. Recombinant proteins can be expressed as fusion proteins with other proteins to make purification easier, for example, as fusion proteins with maltose-binding protein using Escherichia coli as a host (New England Biolabs, USA, vector pMAL series), as fusion proteins with glutathione-S-transferase (GST) (Amersham Pharmacia Biotech, vector pGEX series), or tagged with histidine (Novagen, pET series). The host cells are not limited so long as the cell is suitable for expressing the recombinant proteins. It is possible to use, for example, yeast, various plant or animal cells, insect cells, or such besides the above-described E. coli, by altering the expression vector used. Vectors can be introduced into host cells by a variety of methods known to one skilled in the art. For example, introduction methods using calcium ions (Mandel, M. and Higa, A., Journal of Molecular Biology, 1970, 53, 158-162; Hanahan, D., Journal of Molecular Biology, 1983, 166, 557-580) can be used for introduction into E. coli. Recombinant proteins expressed in the host cells can be purified and recovered from the host cells or the culture supernatant thereof by known methods in the art. When recombinant proteins are expressed as fusion proteins with the afore-mentioned maltose-binding protein or such, affinity purification can be carried out easily. Thus generated proteins encoded by the DNAs of the present invention are also comprised in the present invention.

The proteins of the present invention can be isolated and purified from the plasma membranes of cultured plant cells. The present invention comprises proteins purified by the following steps of:

(1) solubilizing the plasma membrane proteins using surfactants;

(2) binding chitin oligosaccharide elicitor-binding proteins in the solubilized fraction obtained in (1) to a (GlcNAc)₈-APEA derivative; and

(3) eluting the proteins bound in (2).

Such proteins are glycoproteins comprising the sequence from position 1 to 32 of SEQ ID NO: 4 in the Sequence Listing at their N-terminus, and four types of sequences (the sequence from position 139 to 152, the sequence from position 154 to 161, the sequence from position 164 to 176, and the sequence from position 177 to 182 of SEQ ID NO: 4 in the Sequence Listing) in their interior. More specifically, the solubilized plasma membrane is passed through three columns (pre-columns), into which ovalbumin (OVA) was passed first to avoid the loss of proteins due to non-specific adsorption onto the tubes and glass surfaces of columns during the column manipulations. The first and second columns are used to remove substances that adsorb to the sepharose gel support and to dextran, and the third column adsorbs the target proteins of the present invention. The pre-columns, however, are not limited to the two mentioned above. The column used to adsorb the target proteins is a column whose adsorption capacity has been improved substantially by immobilizing (GlcNAc)₈-APEA (aminophenylethylamino) derivatives instead of unmodified chitin oligosaccharides. Next, the proteins of the present invention are eluted from the column. To improve the purity of the target proteins, the column is washed prior to elution with non-elicitor sugars (sugars whose binding modes and structures are similar to those of chitin oligosaccharides, but which do not have activity), and then the target proteins are eluted from the column. By using the above-mentioned isolation and purification methods, large amounts of elicitor-binding proteins can be obtained with a higher purity than in the past.

The proteins of the present invention can be used to prepare antibodies that bind to the proteins. For example, polyclonal antibodies can be prepared by immunizing animals used for immunizations, such as rabbits, with a purified protein of the present invention or a portion thereof, collecting blood after a certain period, and using serum from which clots have been removed. Monoclonal antibodies can be prepared by fusing myeloma cells with antibody-producing cells of animals immunized with the above protein or peptide, isolating monoclonal cells expressing a desired antibody (hybridoma), and recovering the antibodies from the cells. The antibodies thus obtained can be utilized to purify or detect the proteins of the present invention. The present invention comprises antibodies that bind to the proteins of the present invention. The antibodies of the present invention comprise antisera, polyclonal antibodies, monoclonal antibodies, and fragments thereof.

Moreover, the present invention provides vectors comprising the above-mentioned DNAs or nucleic acids, transformed plant cells carrying the vectors, plant transformants comprising the transformed plant cells, plant transformants which are progenies or clones of the plant transformants, and breeding materials of the plant transformants. The plants of the present invention can be used to produce the proteins of the present invention. Furthermore, these plants have the function of defending against diseases such as blast.

When producing plant transformants that express the DNAs of the present invention, the DNAs of the present invention are inserted into appropriate vectors. These are introduced into plant cells and transformed plant cells thus obtained are regenerated.

Moreover, the present invention provides methods for producing the above-mentioned transformed plants, which comprises the steps of introducing a DNA or nucleic acid of the present invention or a vector of the present invention into plant cells, and regenerating plants from the plant cells.

DNAs or nucleic acids of the present invention can be introduced into plant cells by one skilled in the art using known methods such as the Agrobacterium method, electroporation method, or the particle gun method.

The method of Nagel et al., for example, is used for the aforementioned Agrobacterium method (Microbiol. Lett., 1990, 67, 325). According to this method, Agrobacteria are transformed by a recombinant vector and the transformed Agrobacteria are then introduced to plant cells using known methods such as the leaf disc method. The above vector comprises, for example, a promoter to express a DNA of the present invention in a plant subsequent to introduction into that plant. Generally, a DNA of the present invention is placed downstream of such a promoter and, moreover, a terminator is placed downstream of the DNA. Recombinant vectors used for this purpose are suitably selected by one skilled in the art, depending on the introduction method into the plant or type of plant. The above-mentioned promoter may be, for example, a cauliflower mosaic virus-derived CaMV35S promoter or the ubiquitin promoter from maize (Japanese Patent Application Kokai Publication No. (JP-A) H02-79983 (unexamined, published Japanese patent application)).

The above-mentioned terminator may be, for example, a cauliflower mosaic virus-derived terminator or nopalin synthase gene-derived terminator. However, so long as they function as a promoter or terminator in a plant, they are not limited.

Plants into which DNAs or nucleic acids of the present invention are introduced may be explants. Alternatively, cultured cells may be prepared from these plants, and such DNAs or nucleic acids may be introduced into the cultured cells. “Plant cells” in the present invention may be, for example, plant cells from leaves, roots, stems, flowers, seed scutella, as well as calluses and cultured cell suspensions.

In addition, to efficiently select transformed plant cells which have been introduced with a DNA or nucleic acid of the present invention, the above recombinant vector preferably harbors an appropriate selective marker gene, or is introduced into plant cells together with a plasmid vector harboring a selective marker gene. Selective marker genes used for this purpose comprise, for example, the hygromycin phosphotransferase gene, which confers resistance to the antibiotic hygromycin; the neomycin phosphotransferase gene, which confers resistance to kanamycin or gentamycin; and the acetyltransferase gene, which confers resistance to the herbicide, phosphinothricin.

Plant cells into which a recombinant vector has been introduced are plated and cultured on a known selective medium comprising an appropriate selective drug, depending on the type of the introduced selective marker gene. In this way, one can obtain transformed plant cultured cells.

A plant is then regenerated from the transformed cells into which a DNA or nucleic acid of the present invention has been introduced. Regeneration of a plant can be carried out by methods known to one skilled in the art depending on the plant cell type (Toki et al., Plant Physiol., 1995, 100, 1503-1507). Several techniques have already been established to generate transformed rice plants, and those techniques are widely used in the field of the present invention. In the case of rice, for example: (1) plants can be regenerated after genes are introduced into the protoplast using polyethylene glycol (suitable for Indica rice varieties) (Datta, S. K. et al., In Gene Transfer To Plants (Potrykus I and Spangenberg Eds.), 1995, 66-74); (2) plants can be regenerated after genes are introduced into the protoplast using an electric pulse (suitable for Japonica rice varieties) (Toki et al., Plant Physiol., 1992, 100, 1503-1507); (3) plants can be regenerated after genes are directly introduced into the cells using the particle gun method (Christou et al., Bio/technology, 1991, 9, 957-962); or (4) plants can be regenerated after genes are introduced via Agrobacteria (Hiei et al., Plant J., 1994, 6, 271-282). In the present invention, these methods can preferably be used.

The plants regenerated from transformed cells are subsequently cultured in an acclimatization medium. Then, when the acclimatized regenerated plants are grown under normal cultivation conditions, plants can be obtained. Seeds can also be obtained when these plants mature and produce fruit.

The exogenously introduced DNAs or nucleic acids in thus regenerated and grown transformed plants can be confirmed by known methods, such as PCR or Southern hybridization, or by analyzing the nucleotide sequence of the plant's nucleic acids. To extract DNAs or nucleic acids from transformed plants, the known method of J. Sambrook et al. may be used (Molecular Cloning, 2^{nd} edition, Cold Spring Harbor laboratory Press, 1989).

To conduct a PCR analysis of the exogenous gene comprising a DNA of the present invention, which exists in the regenerated plant, an amplification reaction is carried out using nucleic acids that were extracted from the regenerated plant as above as the template. When the nucleic acids of the present invention is a DNA, the amplification reaction may be carried out in a reaction mixture containing, as primers, synthesized oligonucleotides comprising nucleotide sequences appropriately selected according to the DNA's nucleotide sequence. An amplified DNA fragment comprising a DNA sequence of the present invention can be obtained by repeating the denaturation, annealing, and extension reactions for DNA several dozen cycles of the amplification reaction. Each type of amplified DNA fragment can be separated by, for example, electrophoresing the reaction solution comprising the amplified products on an agarose gel and whether the DNA fragments correspond to a DNA of the present invention can then be confirmed.

Having obtained transformed plants in which a DNA of the present invention has been introduced into the chromosomes, one can obtain the plant's progenies by sexual or non-sexual reproduction. Also, it is possible to mass-produce such plants by obtaining breeding materials (such as seeds, fruits, cuttings, stem tubers, root tubers, shoots, calluses, and protoplasts) from the above plant, or its progenies or clones.

The present invention provides pharmaceutical agents for plant disease control. In the present description, “plant disease control” refers to a function of enhancing plant self-defense reactions against invasion of pathogens, such as microbial pathogens and pest insects. More specifically, it refers to functions that are induced when plants are affected with diseases, such as membrane depolarization, production of reactive oxygen, and synthesis of antimicrobial substances such as phytoalexin.

The diseases are, for example, sheath blight, wheat black mold, barley spot blotch, rice brown spot, and are preferably rice blast.

The pharmaceutical agents of the present invention are the DNAs or vectors of the present invention themselves, or compositions that comprise the DNAs or vectors of the present invention. Herein, the “compositions” may comprise various substances. Such substances are not particularly limited so long as a composition accomplishes an objective of the present invention, but examples comprise substances that keep DNAs or vectors stable, substances that assist the introduction of DNAs or vectors into plant cells, substances for increasing an amount so that the DNAs and vectors can be measured easily, and such.

Moreover, the present invention provides methods for plant disease control, where the methods comprise expressing a protein of the present invention in plant cells. Methods for expressing proteins and the like in plant cells are described above.

Furthermore, the present invention provides antisense DNAs against the DNAs of the present invention. Transformed plant cells in which expression of a protein of the present invention is specifically knocked down can be produced by introducing vectors comprising such antisense DNAs into plant cells. The transformed plant cells that are produced can be used to analyze the functions of the proteins of the present invention.

All prior art references cited herein are incorporated by reference into this description.

### EXAMPLES

Hereinbelow, the present invention will be specifically described with reference to Examples, but it is not to be construed as being limited thereto. The Examples were carried out according to the materials and methods described below.

1. Preparation of Cultured Cells and Plasma Membranes

The cultured rice cells (Oryza sativa L. cv. Nipponbare) used in the experiments were maintained by subculturing and growing in a shaking culture using L medium produced by modifying N6 medium (Kuchitsu, K., Kikuyama, M., and Shibuya, N. Protoplasma, 174, 79-81(1993)), in an incubator in the dark at 25° C. and shaken at 150 rpm.

Once every two weeks, the cultured rice cells were strained through a sterilized 1-mm² metal mesh, and the cell masses broken into small pieces were subcultured. In each experiment, the cells were used four or more days after this treatment, considering the effect of stimulation due to straining.

Plasma membranes were prepared from cultured rice cells by obtaining a microsomal fraction (MF) using centrifugation and fractionating this using aqueous biphasic partitioning with dextran/polyethylene glycol, according to the method of Shibuya et al. (Shibuya, N., Ebisu, N., Kamada, Y, Kaku, H., Cohn, J. and Ito, Y Plant Cell Physiol., 37, 894-898(1996)). The obtained plasma membrane fraction was suspended in 1-2 mL of PM buffer, then homogenized by ultrasonication, and stored at −80° C.

2. Elicitor Sugars and Derivatives Thereof

The hexameric or smaller chitin oligosaccharides ((GlcNAc)ₙ) used as elicitors were fine grade samples from Seikagaku Corporation, and heptameric and octameric crab shell-derived chitosan oligosaccharides granted to the present inventors by Yaizu Suisankagaku Industry were reacetylated to prepare each of the heptamers and octamers.

Radioactive iodine, ¹²⁵I, was incorporated into (GlcNAc)₈-APEA derivatives and (GlcNAc)₈-APEA according to the method of Ito et al. (Ito, Y, Kaku, H. and Shibuya N. The Plant Journal, 12(2), 347-356 (1997)).

3. Affinity Labeling

Affinity labeling of elicitor-binding proteins with glutaraldehyde involves using NaCNBH₃ as a catalyst to crosslink amino groups of the ¹²⁵I-(GlcNAc)₈-APEA derivatives with amino groups of protein side chains via glutaraldehyde, and was carried out according to the method of Ito et al. (Ito, Y, Kaku, H. and Shibuya N. The Plant Journal, 12(2), 347-356 (1997)).

Rice plasma membranes, solubilized plasma membrane fractions, or purified CEBiP fractions were reacted with 100 nM (30 pmol) APEA derivative on ice for one hour (fluid volume of 250 μL), then 30 μL of 2.5% glutaraldehyde solution containing 30 μg of NaCNBH₃ was added, and this was reacted at room temperature for 30 minutes. After the reaction, 25 μL of 5 M NaCl and 1.2 mL of MeOH were added, and this was left overnight at −80° C., then centrifuged (15,000 rpm, two hours), and then the precipitated fraction was subjected to SDS electrophoresis. SDS electrophoresis was carried out on a Multiphor II multipurpose electrophoresis apparatus using precast 8-18% polyacrylamide concentration gradient gels (Pharmacia) or on a slab gel electrophoresis apparatus using gels with a polyacrylamide concentration of 10% or 15%, or 5-20% gradient gels (PAGEL, ATTO). After electrophoresis, the gel was fixed in an aqueous 50% methanol/10% acetic acid solution for 30 minutes, the air dried gel or the proteins in the gel were transferred to a PVDF membrane (Millipore Immobilon-PSQ Transfer Membrane), then a bioimaging plate was exposed to the gel for two to seven days. The plates were analyzed using an imaging plate reader and bioimaging analyzer (BAS 2000, Fuji Film) to detect the radiolabeled proteins.

### Example 1

### Purification and amino acid Sequence Analysis of Chitin Elicitor-Binding Proteins

The affinity column used for CEBiP protein purification was prepared by immobilizing each of the (GlcNAc)₈-APEA derivative (75 mg) and glycine onto an activated CH-Sepharose 4B gel carrier (5 g of dry gel), following the manual provided by Pharmacia.

Purification of CEBiP was carried out as follows: the plasma membrane fraction (20.46 mg) was reacted in a TBS buffer (2 mM DTT, 1 mM PMSF, 0.15 M NaCl, 1 mM MgCl₂, 25 mM Tris-HCl buffer (pH 7.0)) containing 0.5% Triton X-100 at 4° C. for one hour, and the supernatant obtained from a table top ultracentrifuge (4° C., 70,000 rpm, one hour) was used as a solubilized plasma membrane fraction.

Surfactants such as TritonX-100 and n-dodecyl-β-maltoside were effective for solubilization of the elicitor-binding protein from the cultured rice cell plasma membrane. 60% of the plasma membrane protein was solubilized by 0.5% Triton X-100, and when binding experiments with ¹²⁵I-labeled elicitor derivatives were performed, approximately 30% of the elicitor-binding protein was collected as an active form. The binding activity of the solubilized fraction showed binding characteristics that corresponded well to the results obtained using the plasma membrane, and results from affinity labeling also confirmed that the target elicitor-binding protein was solubilized while maintaining its activity.

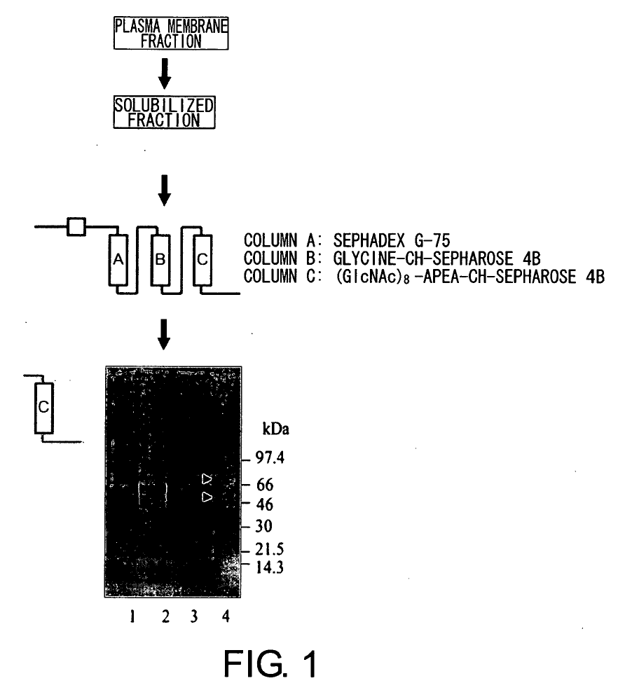

This fraction was applied to three consecutively connected columns (column A: Sephadex G-75 (15 mL); column B: Glycine-CH-Sepharose 4B (10 mL); and column C: (GlcNAc)₈-APEA-CH-Sepharose 4B (11 mL)) (FIG. 1). To avoid losing minute amounts of purified proteins through non-specific adsorption onto the surfaces of the silicon tubes and the glass surfaces of the columns, the connected columns, tubes, and such were washed with 1% ovalbumin (OVA) followed by 0.17 M Glycine-HCl buffer, and then equilibrated using a TBS buffer containing 0.005% Triton X-100 prior to use. Next, the solubilized plasma membrane fraction was fractionated using a set of connected columns, in which two columns (column A and column B) were positioned prior to the column of interest (column C) to remove substances that adsorb to sepharose gel carriers and dextran. In column C, the (GlcNAc)₈-APEA (aminophenylethylamino) derivative was used as a new and high-adsorption capacity carrier for affinity chromatography. Next, the columns were washed with a TBS buffer to remove the unadsorbed substances. Column C, the column of interest, was washed with a mixed solution (1 mg/mL, 20 mL) of the non-elicitor sugars cellohexaose and chitosan hexaose, and then the target protein was eluted using 0.17 M Glycine-HCl buffer (pH 2.3). The eluted fraction was immediately neutralized using a 1 M Tris solution, the total volume was adjusted to 275 μL, then 25 μL of 5 M NaCl and 1.2 mL of MeOH were added to each tube, and this was left overnight at −80° C. The target protein was collected by centrifugation and dissolved to a final volume of 80 μL. 1 μL of this solution was electrophoresed on a precast 8-18% polyacrylamide concentration gradient gel (Pharmacia) using a Multiphor II multipurpose electrophoresis apparatus, then silver staining was carried out. In this way, the bound protein could be isolated and purified in good yield by combining a column that uses APEA derivatives and pre-columns for removing non-specifically-adsorbing substances with effective elution methods. Two bands of 75 kDa and 55 kDa were detected in the fraction purified by this method by SDS electrophoresis using the Multiphor II electrophoresis apparatus (FIG. 1).

The remaining purified protein solution was subjected to SDS electrophoresis through an ATTO 15% polyacrylamide gel, transferred to a PVDF membrane, and stained with CBB, then the band of the target protein was cut out, and analysis of its N-terminal amino acid sequence was performed using an HP241 Protein Sequencer System (Hewlett Packard).

Analysis of the internal chain of the target protein was carried out by the following method: the band was cut out from the gel, soaked in 50 μL of 0.1% SDS, 1 mM EDTA, and 0.1 M Tris-HCl (pH 9.0), then a lysine-specific protease (lysyl endopeptidase, Achromobacter protease I) was added, and digestion was carried out overnight at 37° C. The digest solution was separated through connected columns of DEAE-5PW (1×20 mm; Tosoh, Tokyo) and Mightysil RP-18 (1×50 mm; Kanto Chemical, Tokyo). As solvents, aqueous 0.085% TFA solution was used for A and aqueous 0.075% TFA, 80% CH₃CN solution was used for B. Elution was carried out at 1-12.5-60% B/1-10-86 min., 20 μL/min. The amino acid sequence was clarified and mass spectrometry was carried out on the eluted peaks using a peptide sequencer and MALDI-TOF MS. As a result, the amino acid sequence of the 32 N-terminal residues of the purified target protein were identified and four internal chain amino acid sequences were identified by lysyl endopeptidase treatment.

The two bands detected at 75 kDa and 55 kDa by SDS electrophoresis were both affinity labeled with a ¹²⁵I-labeled (GlcNAc)₈-APEA derivative, and their N-terminal amino acid sequences were identical. Furthermore, peptide map analysis using a protease that specifically cleaves at a lysine suggested that the low-molecular weight band was produced by partial degradation of the C terminus of the 75 kDa protein by endogenous proteases during purification.

As described above, development of method for immobilizing chitin oligosaccharide, not as it is, but as an APEA derivative greatly improved the adsorption capacity of the column support, and when combined with studies on the operating conditions on the whole, the yield was approximately 20 times greater than earlier methods. Under the conditions ultimately set, approximately 1.6% of the elicitor-binding protein present in the plasma membrane is presumed to have been collected. Herein, the N-terminal amino acid residues did not have to be modified artificially during the purification process, and with the help of the increase in yield, the amino acid sequence of the 32 N-terminal residues was clarified. This also allowed separation and purification of peptides that were specifically cleaved with specified peptidases, and this enabled four internal chain amino acid sequences to be clarified by analysis of these peptides.

### Example 2

### Preparation and Purification of Anti-Con A-CEBiP Antibodies

CEBiP is a glycoprotein that binds to concanavalin A (Con A). Therefore, antisera were produced against a rice plasma membrane fraction that binds to a Con A column, and various chromatographies were devised to purify an antiserum (anti-Con A-CEBiP antibodies) against the fraction comprising the present target protein.

Anti-Con A-CEBiP antiserum was prepared by the following procedure: a Con A-Sepharose column was used to prepare Con A-binding total proteins found in the solubilized rice plasma membrane fraction. Using this as an antigen, rabbits were immunized to obtain an anti-Con A-bound fraction antiserum.

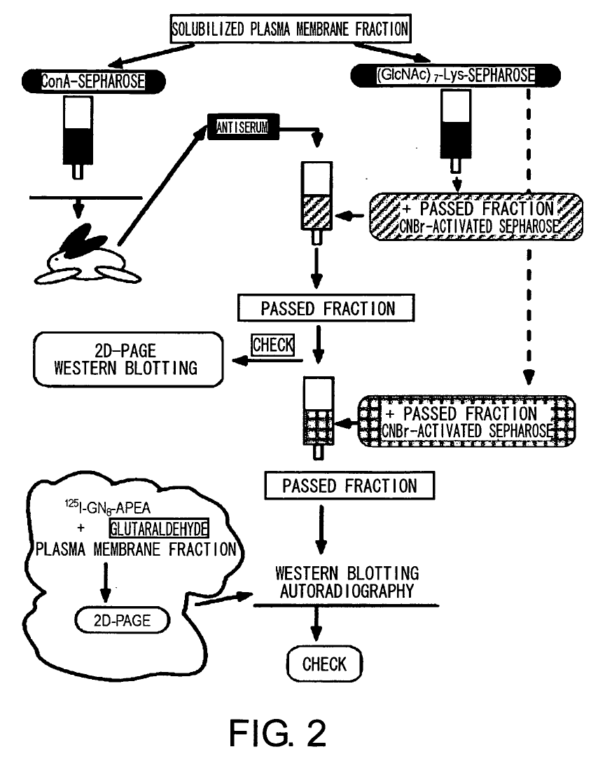

This antiserum was purified by the following method (FIG. 2): columns onto which the fraction that passed through (GlcNAc)₇-Lys-Sepharose and the fraction eluted with non-elicitor sugars (chitosan hexaose and cellohexaose) prepared from the rice plasma membrane fraction were each immobilized were prepared. The anti-Con A-bound fraction antiserum was fractionated using both columns to obtain the anti-Con A-CEBiP antibodies.

The purity of the anti-Con A-CEBiP antibodies was verified by affinity labeling the rice plasma membrane fraction that bound to the Con A column with an ¹²⁵I-labeled elicitor sugar, processing the fraction to a two-dimensional electrophoresis, and then transferring onto a PVDF membrane. The anti-Con A-CEBiP antibodies were used for performing a Western blotting on the transferred membrane. Then, radiolabeled proteins on this membrane were analyzed using a Bio-imaging Analyzer (BAS 2000, Fuji Film). The radiolabeled proteins and the proteins stained by Western Blotting analysis were examined to see if they were the same. Rainbow protein standard (Amersham) and precision protein standard (BIO-RAD) were used as molecular weight standards.

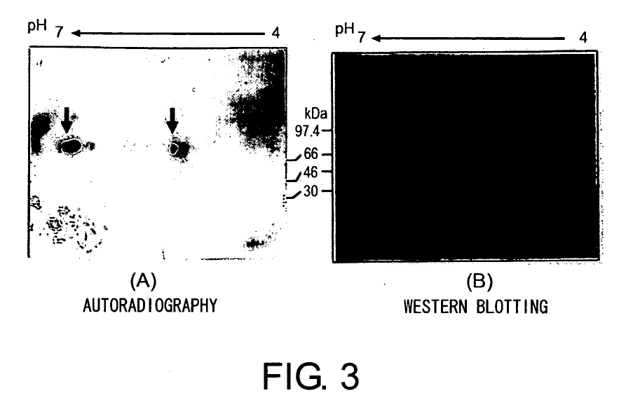

As a result, two major spots from among the three detected by Western Blotting were found to be radiolabeled (FIG. 3). This showed that the anti-Con A-CEBiP antibodies were highly purified against CEBiP.

### Example 3

### Inhibition Analysis of Reactive Oxygen Production Response Using the Antiserum

Protoplasts were prepared from cultured rice cells, and the effect of the anti-Con A-CEBiP antibodies on elicitor-responsive reactive oxygen production was examined.

Rice protoplasts were prepared by the following method:

Cultured rice cells were strained through a metal mesh. Four days later, these cells were placed into 14 mL of 0.1% CaCl₂, 0.02% MES, 9% Mannitol solution (pH 5.6) containing 2% Cellurase RS (Yakult) and 0.05% Pectolyase Y-23, and then gently shaken at 30° C. for six hours. Cells were filtered through a 25 μm nylon mesh, and the cells on the mesh were washed with 20 mL of Washing buffer (0.1% CaCl₂/0.4 M Mannitol) (Nishimura, N., Tanabe, S., He, D.-Y, Yokota, T., Shibuya, N. and Minami, E. Plant Physiol. Biochem., 39, 1105-1110 (2001)). The protoplasts were collected into a 50 mL Falcon tube and recovered by centrifugation at 600 rpm for five minutes. Washing buffer was added, protoplasts were washed several times and suspended in an appropriate amount of R2P medium, and the number of protoplasts was examined using a Thoma counting chamber. After adjusting the number of protoplasts to 2×10⁶ cells/mL, the cells were incubated overnight in the dark at 25° C.

Production of reactive oxygen in rice protoplasts was performed using the luminol method (Schwacke, R. and Hager, A. Planta, 187, 136-141(1992)). Reacted solutions were stored on ice until the measurement. A 50 mM potassium phosphate buffer at pH 7.9 was used as the solvent for luminol and potassium ferricyanide. Potassium ferricyanide was prepared immediately before use, and the luminol solution was stored at low temperature in the dark and was warmed to room temperature before use.

Rice protoplasts (1×10⁶ cells/500 μL) were placed into 2 mL tubes, reacted with preimmune antiserum or anti-Con A-CEBiP antibodies for 30 minutes, then after addition of 1 μL of 100 μg/mL (GlcNAc)₈ or R2P medium, the mixture was stirred slowly for 15 minutes, then centrifuged at 1000 rpm for one minute, and a supernatant was obtained. Protoplasts at a same concentration into which 1 μL of R2P medium or (GlcNAc)₈ was added were used as controls.

25 μL of the reacted solution, 400 μL of 50 mM potassium phosphate buffer (pH 7.9), 25 μL of 1.1 mM luminol, and 50 μL of 14 mM potassium ferricyanide were stirred in a tube, and immediately thereafter, a chemiluminescence count was measured for ten seconds using a luminometer (Turner Designs TD-20/20, Sunnyvale, Calif.). H₂O₂ concentration was calculated by producing a standard curve after measuring the luminescence by a commercially available H₂O₂ solution (aqueous 30% H₂O₂ solution, Wako Pure Chemical Industry).

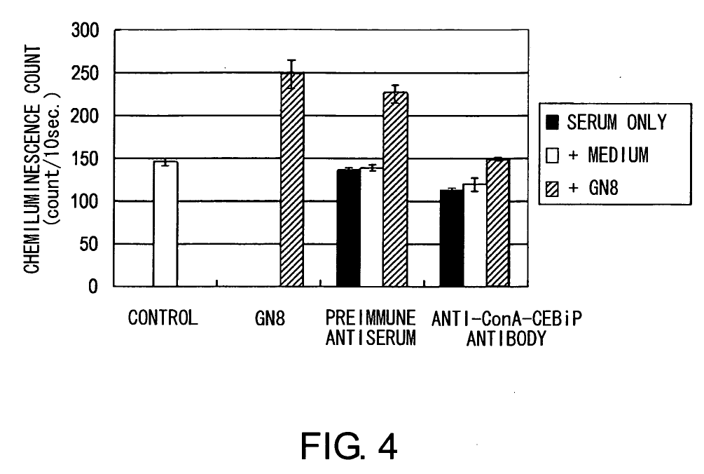

When GlcNAc was added to the protoplasts, production of reactive oxygen increased 1.7 times compared to the control. This increase was not inhibited by a preimmune rabbit serum but almost completely inhibited by a pretreatment with the anti-Con A-CEBiP antibodies (FIG. 4).

These results strongly suggested that this protein is a receptor protein for the chitin oligosaccharide elicitor.

### Example 4

### Screening for CEBiP in a Rice cDNA Library

PolyA RNAs (mRNAs) were isolated from cultured rice cells (Nipponbare) by the phenol-SDS method and oligo dT method, and using this as a template, a cultured rice cell cDNA library was prepared using ZAP-cDNA synthesis kit (STRATAGENE).

Cloning of the genes of the proteins of the present invention was examined using several approaches. Anti-peptide antibodies against the amino acid sequence of the N-terminal 15 residues were produced, the antibodies were purified to high purity, and screening of the cultured rice cell cDNA library was attempted, however, clones comprising the target protein could not be isolated. This was primarily because the amino acid sequence information of 15 residues did not provide enough specificity, and other proteins (other than the membrane fraction) that react with these antibodies may be present in the cultured rice cells. Purified anti-peptide antibodies reacted only with the target protein, showing a single band, as long as microsomal fractions and plasma membrane fractions were used, however, positive clones obtained by screening the rice cDNA library carried sequences that were different from the receptor and similar to known proteins. In addition, since the target protein is synthesized in E. coli as a form comprising a signal peptide in this method, the possibility that reaction with this anti-peptide antibody is difficult due to steric hindrance can be conceived.

In addition, methods comprising the steps of designing degenerate oligonucleotide primers and performing PCR using single stranded circular DNAs produced from cultured rice cell mRNAs and genomic DNAs as templates were considered, however, many bands were amplified and subcloning was difficult. Moreover, methods of directly screening the library using these oligonucleotides as probes were also attempted, however, many non-specific spots were seen and this did not lead to isolation of positive clones.

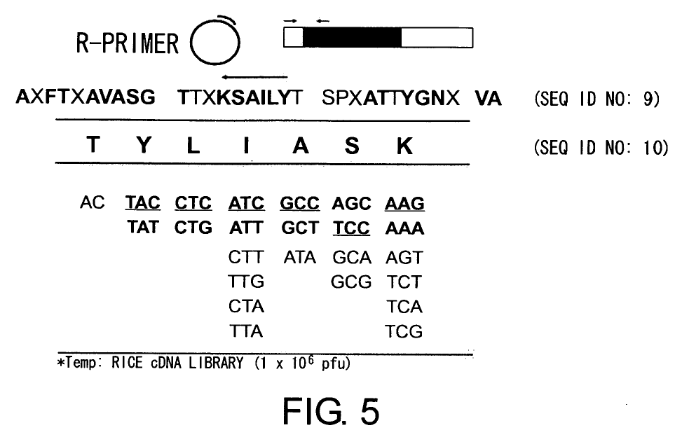

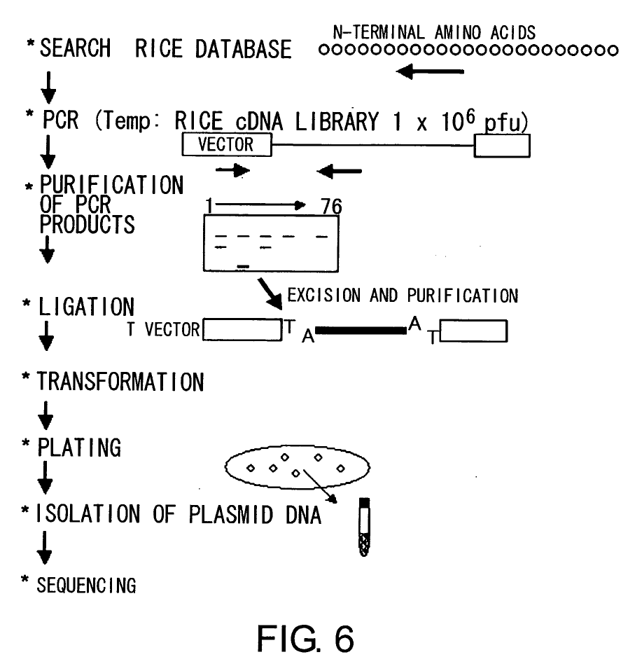

Therefore, the present inventors synthesized 72 types of upstream-oriented primers that correspond to a 7-residue N-terminal amino acid sequence (¹⁴KSAILYT/SEQ ID NO: 10 (reverse direction)) designed by combining frequently used codons of rice (Nipponbare) and performed a PCR using the cultured rice cell cDNA library as a template and each of the synthetic primers and known primers on a vector (FIGS. 5 and 6). As a result of performing PCR using a synthetic primer (TGTAGAGGATGGCGGACTT/SEQ ID NO: 11) and a reverse primer of a vector (GGAAACAGCTATGACCATG/SEQ ID NO: 12), a PCR amplification product corresponding to the target amino acid sequence was successfully obtained. The obtained PCR fragment was subcloned into a TA vector (TA cloning kit, Invitrogen), and the sequence of the PCR fragment was clarified. The size of the PCR fragment obtained herein was 280 bp, and the translated protein sequence started from T, the residue before the signal peptide, and was 49 amino acid residues long.

The DNA corresponding to the N-terminal amino acid sequence of the target protein was cut out from a plasmid, this was used as a probe, and the cultured rice cell cDNA library (4×10⁵ pfu) was screened for the target gene using the 147 bp probe.

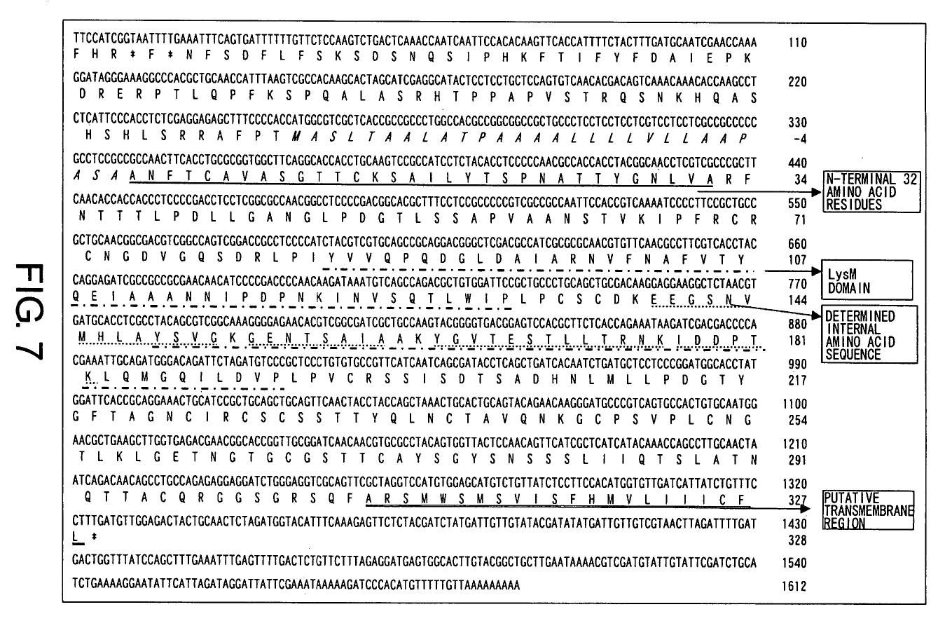

As a result, three cDNA clones whose N-terminal amino acid sequences matched were isolated. These three types of clone all comprised a sequence that completely matched the N-terminal amino acid sequence of the target protein. The mRNA comprised a 28 amino acid residue signal peptide (M-28 to A-1) and a 22 amino acid residue putative transmembrane region (A307 to L328) on the C terminus side (FIG. 7).

### Example 5

### Genomic Southern Blot Analysis

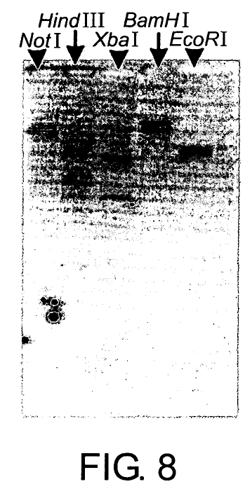

To investigate the number of copies of the target gene, genomic DNAs isolated from rice were treated with various restriction enzymes, electrophoresed on agarose gels, and then subjected to genomic Southern blot analysis using DNAs encoding the target protein as a probe. As a result, a single band was detected for genomic DNAs treated with several types of restriction enzyme (FIG. 8) and this suggested that the target elicitor-binding protein gene exists as a single copy.

This also suggests the possibility that mRNAs encoding the target protein and those encoding the low molecular weight protein with a shared N-terminal sequence are both produced from this single gene through differences in splicing and such.

### Example 6

### Screening for CEBiP in the Rice Genome Library

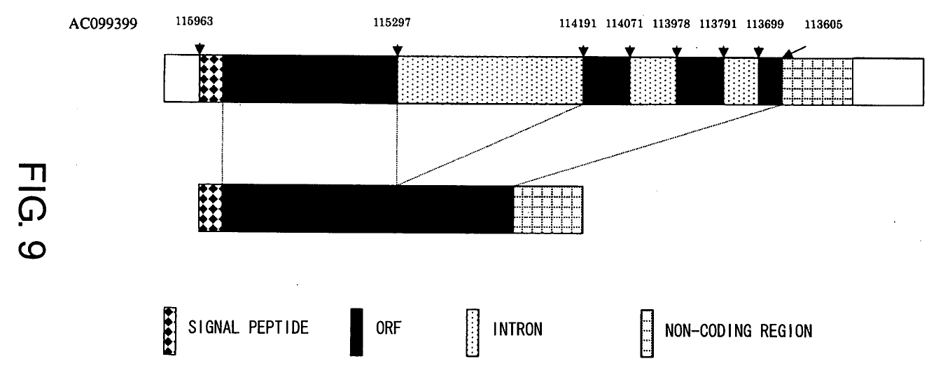

Screening of the rice genome library using probe 1 (a DNA fragment corresponding to the A1 to T181 portion of the amino acid sequence in FIG. 7) allowed isolation of several positive clones. DNAs were isolated from the positive clone with the longest chain length, and after cleaving with a restriction enzyme, each of the fragments was introduced into a plasmid vector, and the sequence with an overall length of 13,095 bp was determined. The target protein portion was completely encoded by this sequence. Later, a gene (AC099399) comprising the genomic DNA analyzed by the present inventors was found from a rice genome database search, and this gene was confirmed to be located on chromosome 3. As a result of matching the sequence of this protein to the genomic DNA sequence, it was revealed that three introns were present in this part of the genomic DNA sequence (FIG. 9) and that mature CEBiP mRNAs are synthesized through splicing of these introns.

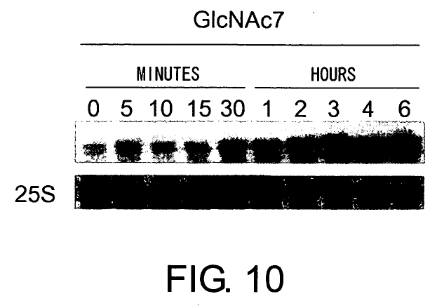

Moreover, the chitin oligosaccharide elicitor was confirmed to induce expression of this target gene (FIG. 10).

Analysis of the translation products of this target gene revealed that the chitin elicitor-binding protein has a molecular weight of 34,640 and comprises 328 amino acid residues comprising a 22-amino acid-residue transmembrane region in the C-terminus side. Furthermore, peptide sequence analysis so far predicted that sugar chains were attached to four of the eleven sites where sugar chains may bind (NXT(S)). Moreover, motif searches revealed that LysM domains which are present in peptidoglycan-binding proteins were present in two sites (Y85 to P131 (sequence of positions 85 to 131 of SEQ ID NO: 4 in the Sequence Listing) and Y149 to P192 (sequence of positions 149 to 192 of SEQ ID NO: 4 in the Sequence Listing) in FIG. 7).

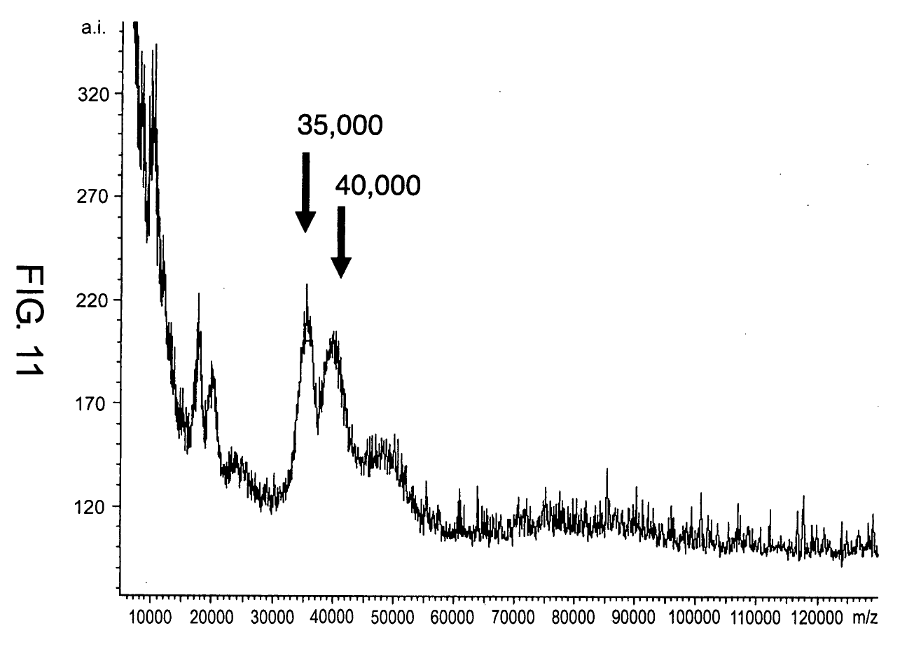

The molecular weight of this target protein was very different from the molecular weight of the 75 kDa protein originally obtained by affinity labeling with ¹²⁵I-labeled elicitor sugar. The present inventors had already reported that the target protein is detected at 75 kDa by electrophoresis using a precast 8-18% polyacrylamide concentration gradient gel (Pharmacia) with a Multiphor II multi purpose electrophoresis apparatus, while the protein is detected between 65 kDa to 67 kDa when using an ATTO slab electrophoresis apparatus (Okada, M., Matsumura, M., and Shibuya, N., J. Plant Physiol. 158, 121-124 (2001)). The rainbow protein standard used was very broad, however, when a more precise molecular weight standard prepared from recombinant proteins was used, the target protein which was until then between 65 kDa to 67 kDa now had a molecular weight of 56 kDa. Furthermore, MALDI TOF-MS (Bruker ReFlex) was used to more accurately measure the molecular weight of CEBiP. Two peaks at 40 kDa and 35 kDa were mainly obtained as a result (FIG. 11). The difference in the molecular weights was considered to come from differences in the sugar chains attached to CEBiP.

### Example 7

### Removal of the CEBiP Sugar Chains by TFMS Treatment

To determine whether the difference in the molecular weight calculated from the gene and the molecular weight estimated from electrophoresis was due to sugar chain additions, the sugar chains of the glycoproteins in the rice plasma membrane proteins were chemically cleaved using trifluoromethanesulfonic acid (TFMS), an SDS-PAGE was carried out followed by a Western Blotting, and then the target proteins were detected using an anti-CEBiP antiserum.

Anti-CEBiP antiserum was produced by the following method:

First, CEBiP was expressed in an E. coli overexpression system. Specifically, a cDNA fragment corresponding to a region excluding the transmembrane region from the target protein was prepared by PCR, this was inserted into a pET16b polyhistidine-labeled vector, and then the sequence was examined. The nucleotide sequence of this cDNA fragment is shown in SEQ ID NO: 7, and the amino acid sequence of the protein encoded by this cDNA is shown in SEQ ID NO: 8. This was introduced into E. coli BL21 and then culture was carried out in LB medium containing 200 μg/mL of carbenicillin at 37° C. for 4.5 hours. E. coli were collected by centrifugation and resuspended in 1 mL of LB medium containing the same concentration of carbenicillin, then 50 μL of this suspension was added to 8 mL of LB medium containing carbenicillin (at a final concentration of 500 μg/mL), and culture was carried out for three hours. E. coli were collected by centrifugation and resuspended in 8 mL of LB medium containing carbenicillin (at a final concentration of 500 μg/mL), IPTG (at a final concentration of 1 mM) was added, and culture was carried out at 30° C. for two hours. Then, bacteria were collected by centrifugation (10,000 rpm, 20° C.) and stored at −80° C.

Next, CEBiP-expressing bacteria were suspended in PBS, disrupted by sonication, and centrifuged to obtain a precipitate fraction. An SDS electrophoresis was performed with this precipitate fraction. The gel band comprising the expressed protein was cut out, PBS was added, and after crushing in a mortar, this was stirred overnight at 4° C. to extract the target protein. An anti-CEBiP antiserum was obtained by immunizing rabbits using this as an antigen.

TFMS treatment and detection of the target protein was carried out by the following method:

Rice plasma membrane fraction (20 μg) was placed into a screw-top vial. After sufficient drying, this was dissolved in 50 μL of trifluoromethanesulfonic acid (TFMS) and allowed to stand for one hour at 0° C. 500 μL of ice-cooled 1 M Tris was added to the reaction solution for neutralization, 275 μL aliquots of this solution were placed into each tube, 27.5 μL of 5 M NaCl and 1.2 mL of MeOH were added, these were allowed to stand overnight at −80° C. and then centrifuged to obtain a precipitate fraction. The precipitate fraction was subjected to SDS electrophoresis and Western blotting and the target protein was detected using the anti-CEBiP antiserum.

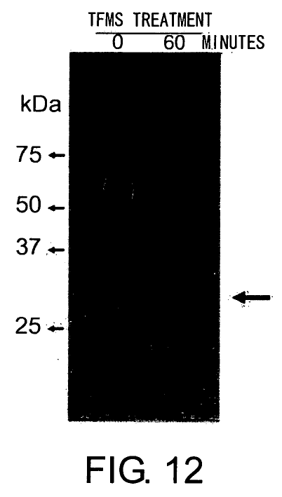

As a result, a positive band was found near 33 kDa (FIG. 12). This molecular weight closely matched the value calculated from the gene.

Okada et al. discovered binding proteins that seem to be identical to CEBiP in the plasma membrane of the leaves and roots of rice (Okada, M., Matsumura, M., and Shibuya, N., J. Plant Physiol. 158, 121-124 (2001)). A similar chitin oligosaccharide-binding protein was found in the plasma membrane obtained from the leaves of wheat, for which chitin oligosaccharide has been reported to induce lignification, however, its molecular size was slightly different. As a result of examining cultured cells of various plants, similar binding proteins were found in the plasma membrane of cultured cells of barley, carrot, and such (Okada, M., Matsumura, M., Ito, Y, and Shibuya, N., Plant Cell Physiol., 43, 505-512 (2002)). These findings suggest that such a chitin-type elicitor recognition system is not unique to rice, but is an evolutionarily conserved system that exists in many plants.

Elucidation of this genetic information is expected to clarify the mechanisms of recognition of microbial pathogen-derived signal molecules (elicitors) by plants and induction of expression of biological defense-related genes through intracellular/intercellular signal transduction pathways, and is considered to contribute to the development of breeds of crops that are resistant to diseases and novel disease control technologies.

### Example 8

### Production of Rice Transformants in Which the Expression Level of CEBiP Gene is Suppressed

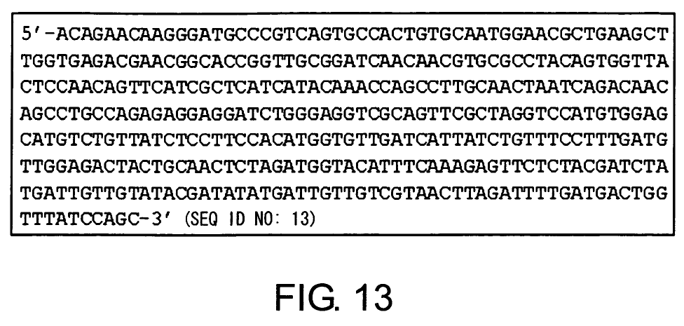

Transformed rice cells in which expression of CEBiP gene was specifically knocked down by the RNAi method were produced by the Agrobacterium method to prove that CEBiP in rice cells functions as a chitin elicitor receptor. The binary vector for transformation was constructed using the Gateway system method. First, PCR was performed with the CEBiP gene clone as a template, and using an upstream primer (5′-CACCACAGAACAAGGGATGCCCGT-3′/SEQ ID NO: 14), a downstream primer (5′-GCTGGATAAACCAGTCATCAAAAT-3′/SEQ ID NO: 15), and KOD-Plus DNA polymerase (TOYOBO) to amplify a 385 bp CEBiP gene fragment (FIG. 13/SEQ ID NO: 13). This was cloned into an entry vector using a pENTR/D-TOPO Cloning kit (Invitrogen) (pENTR-CEBiP-RNAi).

pENTR-CEBiP-RNAi was treated with the restriction enzyme MboII, then mixed with a binary vector for the Gateway system, pANDA (allotted by Prof. Ko Shimamoto at the Nara Institute of Science and Technology; Miki D. & Shimamoto K. Plant cell physiology, 45, 490-495, 2004), and using a Gateway LR Clonase Enzyme Mix (Invitrogen) the pANDA-CEBiP-RNAi binary vector, in which two repeats of the CEBiP gene fragment were introduced in opposite directions on both sides of a GUS gene fragment, was constructed.

Rice (cultivar: Nipponbare) was transformed by an ultra-fast rice transformation technique (WO 01/06844) using an Agrobacterium tumefaciens EHA105 strain into which pANDA-CEBiP-RNAi has been introduced by electroporation. Transformed rice cells were selected by subculturing four or more times every ten days or so with N6D Gel-Rite medium containing carbenicillin and hygromycin (selection medium) to produce transformed rice callus lines (CEBiP-RNAi lines).

The expression level of the CEBiP gene was analyzed by detecting CEBiP proteins in each rice callus by Western blot analysis using an anti-CEBiP rabbit antiserum and detecting the transcription level of the CEBiP gene by RT-PCR. Specifically, 150 μL of a sample solution for SDS was added to 50 mg each of non-transformed (NT) rice calli and CEBiP-RNAi rice calli, these were heated at 100° C. for five minutes, and then ground using a homogenizer. 20 μg of each protein sample from the supernatant from centrifugation was separated by SDS electrophoresis, then the proteins were transferred onto a PVDF membrane, and CEBiP was detected using an anti-CEBiP antiserum. Moreover, RNAs were extracted from the NT rice calli and five CEBiP-RNAi rice callus lines (100 mg) using RNeasy Plant Mini Kit (QIAGEN). cDNAs were synthesized from 100 ng of each of the obtained RNAs using Oligo(dT)₂₀ and reverse transcriptase from the TOYOBO ReverTra Ace-α-Kit, and PCR was performed with one fourth the amount of cDNA as a template, and using an upstream primer (5′-CACCCTACAGTGGTTACTCCA-3′/SEQ ID NO: 16), a downstream primer (5′-TCCTATCTAATGAATATTCC-3′/SEQ ID NO: 17), and KOD-Plus DNA polymerase (TOYOBO). PCR was carried out by performing 25 cycles of 98° C. for ten seconds, 45° C. for two seconds, and 74° C. for 30 seconds. Thereafter, electrophoresis was carried out with a 1.5% agarose and the amount of DNA fragments derived from the transcripts of the CEBiP gene was examined. As controls, the amount of ubiquitin gene transcripts and the amount of transcripts derived from the GUS gene fragment in the pANDA-CEBiP-RNAi vector were similarly examined. The upstream primer (5′-TACCCGCTTCGCGTCGGCAT-3′/SEQ ID NO: 18) and downstream primer (5′-TGCTTCCGCCAGTGGCGCGA-3′/SEQ ID NO: 19) were used for the GUS gene, and the upstream primer (5′-CCAGTAAGTCCTCAGCCATGGA-3′/SEQ ID NO: 20) and downstream primer (5′-GGACACAATGATTAGGGATCAC-3′/SEQ ID NO: 21) were used for the ubiquitin gene.

The expression level of the CEBiP gene was quantified by real-time PCR measurements. The template used for real-time RT-PCR was a sample prepared by adding Oligo dT primers to RNAs (250 ng) extracted from cultured cells that were induced from NT rice calli and CEBiP-RNAi #3 and #6 rice calli and performing reverse transcription using Superscript I (Nippon Gene). Real-time PCR was carried out on the LightCycler Instrument by adding LightCycler-Fast Start DNA Master SYBR Green I (Roche Diagnostics Japan) and two types of CEBiP primers: upstream primer (5′-ATGGAACGCTGAAGCTTGGTGAGA-3′/SEQ ID NO: 22); and downstream primer (5′-CTCATCCTCTAAAGAACAGAGTCA-3′/SEQ ID NO: 23). The amount of rice ubiquitin gene transcription product was used as a control, for which (5′-CCAGTAAGTCCTCAGCCATGGA-3′/SEQ ID NO: 20) and (5′-GGACACAATGATTAGGGATCAC-3′/SEQ ID NO: 21) were used as primers. The quantitative PCR program consisted of one cycle of 95° C. for ten minutes, and then 45 cycles of 95° C. for 15 seconds, 60° C. for five seconds, and 72° C. for ten seconds, and following the protocol provided by Roche, the obtained results were corrected using the amount of ubiquitin gene transcripts, and the CEBiP content in each of the cultured cells was then calculated.

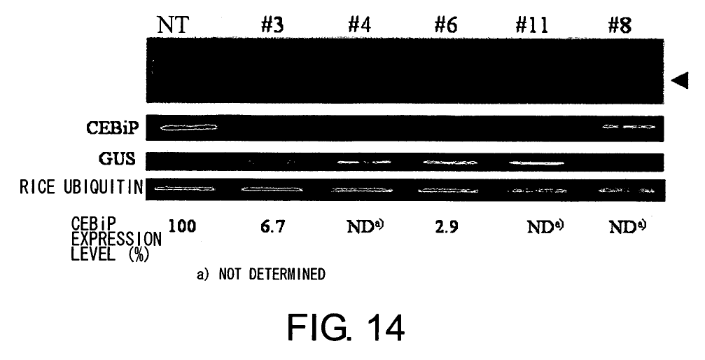

As a result, CEBiP proteins were not detected in four CEBiP-RNAi rice cell lines (#3, #4, #6, and #11), whereas they were detected in NT and in CEBiP-RNAi #8-derived rice cells by a Western blotting using an anti-CEBiP rabbit antiserum (FIG. 14). Similarly, expression of transcripts derived from the CEBiP gene was not confirmed in the four CEBiP-RNAi rice cell lines, whereas expression of the gene was observed in both NT and CEBiP-RNAi #8 rice cells by the RT-PCR method, supporting the results of the Western blot analysis. Transcripts derived from the GUS gene fragment, which serves as an index for gene introduction, were confirmed in these four CEBiP-RNAi rice cell lines and transcripts derived from the rice ubiquitin gene used as a control were detected in all rice cells. From these results, the expression level of the CEBiP gene was judged to be suppressed in the four transformed rice callus lines.

Quantification of the expression level of CEBiP by real-time PCR revealed that, when the CEBiP expression level in NT rice cells is defined as 100%, that of CEBiP-RNAi #3 rice cells is 6.7% and that of CEBiP-RNAi #6 rice cells is 2.9%. Therefore, the CEBiP expression levels of the two CEBiP-RNAi rice cells were considered to be suppressed by 93.3% and 97.1%, respectively, compared to NT rice cells (FIG. 14). In the following Example, #3 and #6 were used as RNAi-suppressed lines.

### Example 9

### Analysis of Reactive Oxygen Production Due to Sugar Elicitors in CEBiP-RNAi Cultured Rice Cells

Reactive oxygen production due to sugar elicitor treatment in NT and CEBiP-RNAi cultured rice cells was analyzed as follows: NT rice calli and CEBiP-RNAi rice calli were placed into 100 mL Erlenmeyer flasks containing 30 mL of N6 medium. After culturing at 27° C. for one week, the cells were strained, 0.6 mL of cells were transferred to 30 mL of fresh medium and cultured for another four days. 60 mg or 100 mg of each of the cultured cells were placed into a 2 mL tube, 1 mL of fresh N6 medium was added, and the cells were cultured overnight. (GlcNAc)₈ (100 ng/mL), Pseudomonas aeruginosa-derived lipopolysaccharide (LPS, 50 μg/mL, Sigma), and (GlcNH₂)₇ (2 μg/mL) were used as elicitors. An elicitor was added to NT and CEBiP-RNAi cultured rice cells, treatment was carried out for 30 minutes or two hours, then 25 μL of the medium solution was sampled for reactive oxygen measurements. As a control, a same amount of distilled water (DW) was added, and similar treatments were carried out. Reactive oxygen was measured by adding 25 μL of the reacted solution, 400 μL of 50 mM potassium phosphate buffer (PK, pH 7.9), 25 μL of 1.1 mM luminol, and 50 μL of 14 mM potassium ferricyanide to a tube, mixing, then immediately measuring chemiluminescence counts for ten seconds in a luminometer (Turner Design TD-20/20, Sunnyvale, Calif.).

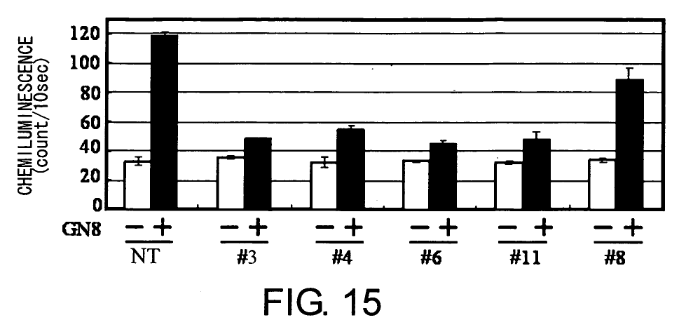

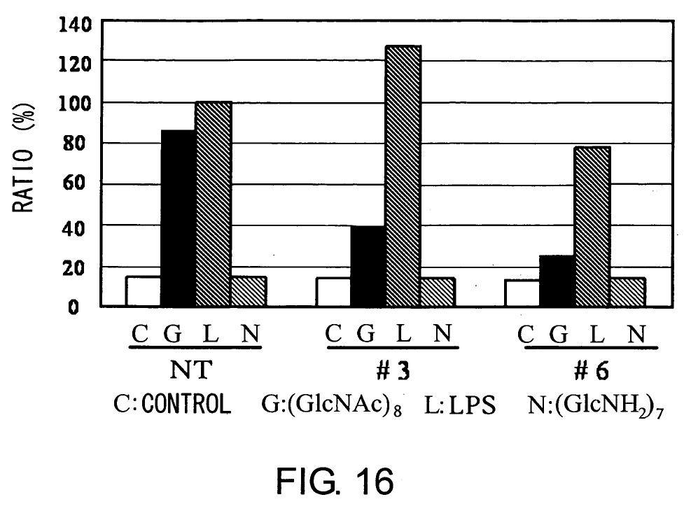

When the amount of reactive oxygen produced due to the (GlcNAc)₈ elicitor was investigated in each type of rice cell, production in CEBiP-RNAi rice cells was found to be suppressed by 74% to 86% compared to NT rice cells (FIG. 15). On the other hand, production of reactive oxygen was recently found to be induced in rice cells treated with LPS (Desaki et al., unpublished), and when CEBiP-RNAi cell lines were treated with LPS, induction of reactive oxygen was observed similarly to NT rice cells. When the amount of reactive oxygen produced in NT rice cells due to LPS treatment was defined as 100%, the production of reactive oxygen seen in NT rice cells due to (GlcNAc)₈ treatment was approximately 86.3%, whereas in two CEBiP-RNAi rice cell lines, the production was suppressed by 60% to 75% (FIG. 16). Non-responsiveness to the non-elicitor carbohydrate (GlcNH₂)₇ was maintained in both CEBiP-RNAi lines. These results strongly suggested that CEBiP is an important protein involved in signal transduction as a receptor that specifically recognizes the chitin elicitor.

### Example 10

### Analyses Using Microarrays

An oligoarray (Agilent) produced from a rice full-length cDNA database was used for the microarray analyses. For the preparation of Cyanine 3 (Cy3)- and Cyanine 5(Cy5)-labeled cRNAs, RNAs isolated from each of the cultured cells of NT and two kinds of CEBiP-RNAi cultured rice cells treated with (GlcNAc)₈ for two hours ((GlcNAc)₈-NT, (GlcNAc)₈-CEBiP-RNAi #3, and (GlcNAc)₈-CEBiP-RNAi #6) as well as of cultured cells treated with water as controls were used. Methods for Cy3- and Cy5-labeling and methods for hybridizing the Cy3- and Cy5-labeled samples with slide glasses onto which 22K rice oligoprobes have been attached were carried out according to the protocol from Agilent. The combinations of hybridization reagents that were used were [1] Cy5-labeled (GlcNAc)₈-NT/Cy3-labeled NT; [2] Cy5-labeled (GlcNAc)₈-CEBiP-RNAi #3/Cy3-labeled CEBiP-RNAi #3; and [3] Cy5-labeled (GlcNAc)₈-CEBiP-RNAi #6/Cy3-labeled CEBiP-RNAi #6. Furthermore, experiments in which the labeling reagents were swapped (the labeled forms were interchanged) were also performed. The obtained results were analyzed at http://tos.nias.affrc.go.jp/cgi-bin/tos17/array.cgi.

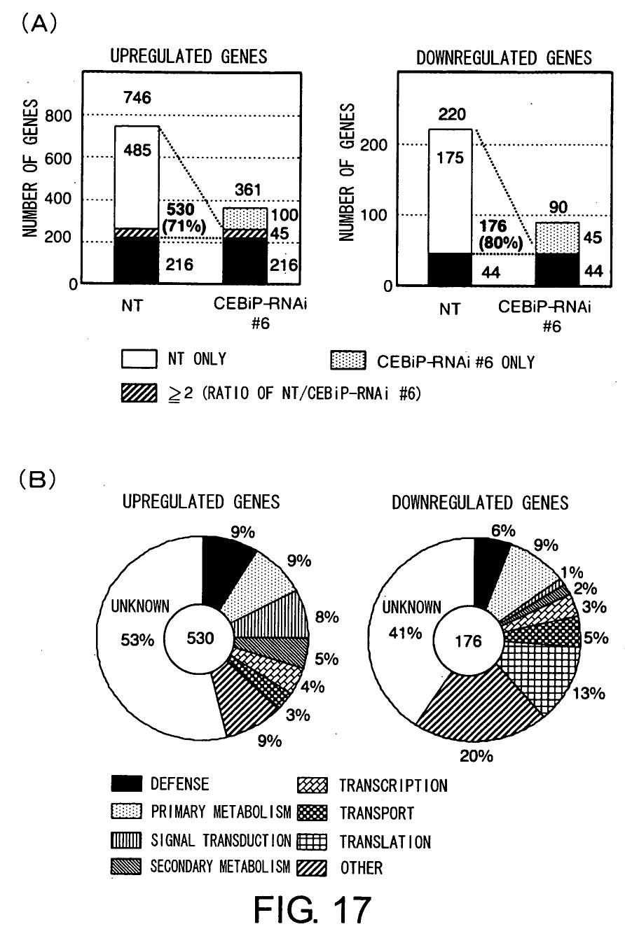

As a result, in the (GlcNAc)₈-NT/NT area, 746 genes whose expression level increased and 220 genes whose expression level decreased were identified (FIG. 17A). In contrast, in the (GlcNAc)₈-CEBiP-RNAi #6/CEBiP-RNAi #6 area, 361 genes whose expression level increased, and 90 genes whose expression level decreased were identified. In CEBiP-RNA #6 cells, 530 of the 746 genes in the (GlcNAc)₈-NT/NT area whose expression level increased, which corresponds to 71% of these genes, showed a suppression of two times or more (45 genes) or loss of elicitor responsiveness compared to the results obtained from non-transformant rice (FIG. 17A). Of these known gene group, approximately 50% were involved in defense, primary metabolism, signal transduction, secondary metabolism related to phytoalexin synthesis, and such (FIG. 17B).

Representative genes were selected from among these genes, and are shown in Table 1. In CEBiP-RNAi cells, among the elicitor responsive genes, elicitor responsiveness was lost for: the CEBiP gene (AK073072) as well as genes related to phytoalexin synthesis such as Caffeoly-CoA 3-O-methyltransferase (AK071482) and PAL (AK068993); genes related to the shikimic acid pathway such as shikimate kinase (AK066687); genes related to ethylene biosynthesis such as 1-aminocyclopropane-1-carboxylate oxidase (AK058296); genes related to lignin degradation such as Laccase (AK103094); and genes related to the MAPK pathway. Therefore, this suggested that these groups of genes are downstream of the CEBiP protein. Interestingly, genes of Harpin-induced protein (Hin, AK068115, AK063651) and Pirin (AK105971), which are considered to be related to cell death, and a negative regulatory gene of cell death (Spl 11, AK105835) similarly lost elicitor responsiveness. Therefore, these groups of gene were suggested to be regulated downstream of CEBiP.

Moreover, one hundred of the 361 genes (approximately 28%) in the (GlcNAc)₈-CEBiP-RNAi #6/CEBiP-RNAi #6 area whose expression level increased due to elicitor treatment had increased expression levels compared to NT cells (FIG. 17A), and among these genes were genes for Ribosome inactivating protein 2 (AK103707), NADPH HC toxin reductase (AK065812), Calreticulin (AK060834) and so on (Table 1).

On the other hand, 176 genes, corresponding to 80% of the genes whose expression level decreased in the (GlcNAc)₈-NT/NT area, showed a suppression of two times or more or a loss compared to the results of non-transformants in the (GlcNAc)₈-CEBiP-RNAi #6/CEBiP-RNAi #6 area (FIG. 17A), and these genes were mainly genes involved in transport, transcription, and defense (FIG. 17B). Furthermore, in 216 genes whose expression level increased due to elicitor treatment and 44 genes whose expression level decreased due to the treatment, equivalent genetic responses were observed in both non-transformant rice and CEBiP-RNAi #6 rice cells. Genes whose expression level increased comprised NAC transcription factor (AK067690) and Small GTP-binding protein (AK066784); genes whose expression level decreased comprised Expansin (AK100959) and Cysteine proteinase (AK072235). Similar trends were also observed in CEBiP-RNAi #3 rice cells.

TABLE 1

| ACCESSION | | FOLD INCREASE | |
|---|---|---|---|
| NUMBER | CHARACTERISTICS | NT* | RNAi #6 |
| AK063651 | Harpin induced protein | 24.7 | N.D^{a} |
| AK101431 | Patatin-like protein | 19.6 | N.D |
| AK105971 | Pirin | 20.6 | N.D |
| AK071482 | Caffeoyl-CoA 3-O-methyl- | 11.3 | N.D |
| | transferase | | |
| AK069456 | Peroxidase | 10.9 | N.D |
| AK066687 | Shikimate kinase | 9.5 | N.D |
| AK058296 | 1-aminocyclopropane-1-car- | 6.6 | N.D |
| | boxylate oxidase | | |
| AK073072 | CEBiP | 2.6 | N.D |
| AK068115 | Harpin induced protein | 43.1 | 10.1 |
| AK068993 | PAL | 48.4 | 2.9 |
| AK103094 | Laccase | 10.0 | 2.1 |
| AK067690 | NAC Transcription factor | 2.4 | 3.4 |
| AK066784 | Small GTP-binding protein | 2.1 | 2.5 |
| | (rab5A) | | |
| AK103707 | Ribosome inactivating | N.D | 27.8 |
| | protein 2 | | |
| AK065812 | Zea mays NADPH HC toxin | N.D | 16.0 |
| | reductase | | |
| AK058891 | β-1.3 glucanase | 4.1 | 11.4 |
| AK060834 | Calreticulin | 3.3 | 14.2 |
| AK060423 | Alanine:glyoxylate | 11.2 | N.D^{a} |
| | aminotransferase like | | |
| | protein | | |
| AK071598 | Lipid transfer protein | 10.0 | N.D |
| AK060625 | GDSL-motif lipase/hydrolase | 6.4 | N.D |
| AK060582 | Replication protein | 4.6 | 2.8 |
| | A1(Os-RPA1) | | |
| AK102192 | β-Galactosidase | 3.1 | 2.8 |
| AK100959 | beta-Expansin(EXPB3) | 5.5 | 5.4 |
| AK072235 | Cysteine proteinase | 4.3 | 5.5 |

^{a}N.D, NOT DETECTED

According to the series of microarray experiments, 70 percent or more of the genes that responded to chitin oligosaccharide elicitors in cultured rice cells lost responsiveness when the expression level of CEBiP decreased; therefore, CEBiP was confirmed to be a receptor protein for chitin oligosaccharide elicitors, and a protein that plays an important role in elicitor signal transduction.

### INDUSTRIAL APPLICABILITY

Elicitors are known to induce various biological defense-related genes, and to cause defense reactions in plants. Since the proteins of the present invention are considered to be elicitor receptors, overexpressing the proteins of the present invention can induce various biological defense responses. Therefore, the proteins of the present invention may be used to provide novel disease control technologies against diseases such as blast. Furthermore, proteins similar to the proteins of the present invention exist not only in rice, but also in many other plants. Elucidation of the present genetic information is expected to reveal the mechanisms by which plants recognize microbial pathogen-derived signal molecules (elicitors), and induce the expression of biological defense-related genes through intracellular/intercellular signal transduction pathways, and may contribute to the development of many disease-resistant crop breeds, comprising rice.

1. A DNA encoding a plant protein that has a binding activity to a chitin oligosaccharide elicitor, wherein the DNA is any one of (a) to (d):
(a) a DNA comprising the nucleotide sequence of SEQ ID NO: 1 or 3;
(b) a DNA that hybridizes with a DNA comprising the nucleotide sequence of SEQ ID NO: 1 or 3;
(c) a DNA encoding a protein comprising the amino acid sequence of SEQ ID NO: 2 or 4; and
(d) a DNA encoding a protein comprising an amino acid sequence with a substitution, deletion, addition, and/or insertion of one or more amino acids in the amino acid sequence of SEQ ID: NO: 2 or 4.

2. The DNA of claim 1, wherein the plant is rice.

3. A protein encoded by the DNA of claim 1.

4. A vector comprising the DNA of claim 1.

5. A transformed plant cell that carries the DNA of claim 1.

6. A plant transformant comprising the transformed plant cell of claim 5.

7. The plant transformant of claim 6, which is derived from rice.

8. A plant transformant that is a progeny or a clone of the plant transformant of claim 6.

9. A breeding material of the plant transformant of claim 6.

10. A method for producing the plant transformant, wherein the method comprises the steps of introducing the DNA of claim 1 into a plant cell, and regenerating a plant from the plant cell.

11. A pharmaceutical agent used to control a plant disease, wherein the agent comprises the DNA of claim 1.

12. The pharmaceutical agent of claim 11, wherein the plant is rice.

13. The pharmaceutical agent of claim 12, wherein the disease is blast.

14. A method for controlling a plant disease, wherein the method comprises the step of expressing the protein of claim 3 in a cell of a plant.

15. The method of claim 14, wherein the plant is rice.

16. The method of claim 15, wherein the disease is blast.
